(19) United States
(12) Patent Application Publication
(10) Pub. No.: US 2023/0295615 A1
(43) Pub. Date: Sep. 21, 2023

(54) Targeted Sequence Insertion Compositions and Methods

(71) Applicant: The Jackson Laboratory, Bar Harbor, ME (US)

(72) Inventors: Albert Cheng, Unionville, CT (US); Nathaniel Jillette, Farmington, CT (US)

(21) Appl. No.: 18/040,939

(22) PCT Filed: Aug. 6, 2021
(86) PCT No.: PCT/US2021/044924

§ 371 Date: Feb. 7, 2023

Related U.S. Application Data
(60) Provisional application No. 63/062,743, filed on Aug. 7, 2020.

(51) Int. Cl. C12N 15/11; C12N 9/12; C12N 9/22

(52) U.S. Cl. CPC C12N 15/11; C12N 9/1276; C12N 9/22; C12N 2310/20

(57) ABSTRACT

The invention described herein provides compositions and reagents for integrating large sequences (e.g., 150 bp or more) into a selected target DNA sequence via prime editing.

### REFERENCE TO RELATED APPLICATIONS

This application claims the benefit of the filing date of U.S. Provisional Patent Application No. 63/062,743, filed on Aug. 7, 2020. The entire contents of the foregoing application are incorporated herein by reference.

### GOVERNMENT SUPPORT

The invention described herein was made with U.S. government support under Grant No. R01-HG009900, awarded by NHGRI/NIH (the National Institute of Health). The U.S. government has certain rights in the invention

### BACKGROUND OF THE INVENTION

CRISPR/Cas technology has provided great power in editing the genome. While indel-mediated knockout of genes is largely efficient with due to the ubiqutous the non-homologous end joining (NHEJ) repair pathway that is mediated following the induction of double-stranded breaks (DSBs), the ability to make precise editsing or to incorporate substantial and accurate changes typically relies on less efficient pathways homologous recombination happening at the much lower efficiency, and is restricted mostly to dividing cells. Furthermore, the reliance on DSBs means that off-target cutting by Cas9 site-specific endonucleases might induce unwanted perturbations in the genome.

Prime editing is a recent advance in the CRISPR/Cas toolbox, in which a Cas9 nickase generates a single-stranded nick at a target region; the nicked strand subsequently acts as primer annealing to an extended prime editing guide RNA (pegRNA) which serves as template for reverse transcription. Sequence changes or insertions are encoded in the template region of pegRNA, and are incorporated at the target region. Prime editing allows addition of sequences without DSBs in non-dividing cells, thus improving the safety and scope of CRISPR/Cas genome editing.

Prime editing has been shown to insert sequences up to 44 bp. However, longer sequence insertions might not be achievable using this technology.

### SUMMARY OF THE INVENTION

One aspect of the invention provides a polynucleotide comprising: (1) a DNA-targeting sequence that is complementary to the target strand (TS) of a double-stranded target DNA sequence, the target strand is complementary to the non-target strand (NTS) of the double-stranded target DNA sequence; (2) a binding sequence for a CRISPR-Cas system effector enzyme; (3) an integrase or recombinase recognition sequence or complement thereof; and, (4) a primer binding sequence complementary to a primer sequence.

In some embodiments, the CRISPR-Cas system effector enzyme is a Type II or Type V Class II CRISPR-Cas system effector enzyme.

In some embodiments, the primer sequence comprises the 3′ end resulting from the cleavage of the non-target strand by (e.g., the RuvC or RuvC-like nuclease activity of) the effector enzyme, and/or when the polynucleotide is complexed with the effector enzyme via the binding sequence and guides the cleavage of the non-target strand by (e.g., the RuvC or RuvC-like nuclease activity of) the effector enzyme.

In a related aspect, the invention provides a pegRNA (primer editing guide RNA) comprising a recombinase or integrase recognition sequence (or complement thereof), wherein the recombinase or integrase recognition sequence (or complement thereof) is positioned on the pegRNA for insertion into a target DNA of the pegRNA.

In certain embodiments, the effector enzyme is a Type II Class II CRISPR-Cas system effector enzyme.

In certain embodiments, the effector enzyme is a Cas9, such as SpCas9 from 20 Streptococcus pyogenes, SaCas9 from Staphylococcus aureus, StCas9 from Streptococcus thermophilus, NmCas9 from Neisseria meningitidis, FnCas9 from Francisella novicida, CjCas9 from Campylobacter jejuni, ScCas9 from Streptococcus canis, or a variant thereof (such as eSpCas9, SpCas9-HF1, and xCas9).

In certain embodiments, the effector enzyme lacks HNH nuclease activity.

In certain embodiments, the effector enzyme is a Type V Class II CRISPR-Cas system effector enzyme that lacks HNH nuclease activity.

In certain embodiments, the effector enzyme is Cpf1, C2c1, or C2c3, optionally, the effector enzyme is a nickase with only RuvC nuclease activity.

In certain embodiments, the recombinase is a Cre recombinase, a Hin recombinase, a Tre recombinase, or an FLP recombinase.

In certain embodiments, the integrase is a phage-encoded serine integrase (such as R4, φC31, φBT1, Bxb1, SPBc, TP901-1, Wβ, FC1, φK38, RV, A118, BL3, MR11, TG1 and φ370). In certain embodiments, the integrase is a transposase (such as Tn3, Tn5, Tn7, piggyBac, SleepingBeauty, or mos1).

In certain embodiments, the integrase is Bxb1 or φC31.

In certain embodiments, the polynucleotide is an RNA.

In certain embodiments, the DNA-targeting sequence is about 11-13 bases in length, about 14-20 bases in length, about 21-72 bases in length, or about 32-38 bases in length.

In certain embodiments, the DNA-targeting sequence is complementary to the target strand of the double-stranded target DNA sequence over about 12-22 nucleotides (nts), about 14-20 nts, about 16-20 nts, about 18-20 nts, or about 12, 14, 16, 18, or 20 nts (preferably, the complementary region comprises a continuous stretch of 12-22 nts, preferably at the 3′ end of the DNA-binding sequence).

In certain embodiments, the DNA-targeting sequence is at least about 60%, 70%, 80%, 85%, 90%, 95% or more complementary to the target strand.

In certain embodiments, the DNA-targeting sequence has a 5′ end nucleotide G.

In certain embodiments, the polynucleotide further comprises a linker sequence linking the DNA-targeting sequence to the binding sequence.

In certain embodiments, the binding sequence comprises a hairpin structure.

In certain embodiments, the binding sequence is about 37-47 nt, or about 42 nt.

In certain embodiments, the integrase or recombinase recognition sequence comprises recognition sequence for two different integrases or recombinases.

In certain embodiments, the integrase or recombinase recognition sequence comprises any one of SEQ ID NOs: 1, 2, 4, 5, 9, 11, or 12.

In certain embodiments, the complementary sequence is at least about 6, 8, 10, 12, 14, 15, 16, 18, or 20 bases in length.

In certain embodiments, the complementary sequence is at the 3′ end of the polynucleotide.

In certain embodiments, the target DNA sequence is at, within, or adjacent to a target gene of interest (GOI).

In certain embodiments, GOI is a defective gene, a disease gene, or a wild-type or mutant gene desired to be inactivated.

In certain embodiments, the target DNA sequence is within the first intron of the GOI.

In certain embodiments, the target DNA sequence comprises or is adjacent to a transcription regulatory element.

In certain embodiments, the transcription regulatory element comprises one or more of: core promoter, proximal promoter element, enhancer, silencer, insulator, and locus control region.

In certain embodiments, the target DNA sequence comprises or is adjacent to a telomere sequence, a centromere, or a repetitive genomic sequence.

In certain embodiments, the target DNA sequence comprises or is adjacent to a genomic marker sequence (or a genomic locus of interest).

Another aspect of the invention provides a vector encoding any one of the polynucleotide of the invention.

In certain embodiments, transcription of the polynucleotide is under the control of a constitutive promoter, or an inducible promoter.

In certain embodiments, the vector is active in a cell from a mammal (a human; a non-human primate; a non-human mammal; a rodent such as a mouse, a rat, a hamster, a Guinea pig; a livestock mammal such as a pig, a sheep, a goat, a horse, a camel, cattle; or a pet mammal such as a cat or a dog); a bird, a fish, an insect, a worm, a yeast, or a bacterium.

Another aspect of the invention provides a plurality of vectors of the invention, wherein two of the vectors differ in the encoded polynucleotides in their respective DNA-targeting sequences, binding sequences, integrase or recombinase recognition sequences, and/or complementary sequences.

Another aspect of the invention provides a complex comprising: (a) any one of the polynucleotide of the invention, and, (b) a fusion protein comprising: (i) a Type II or Type V Class II CRISPR-Cas system effector enzyme lacking HNH nuclease activity; and, (ii) a reverse transcriptase; wherein the polynucleotide is complexed with the effector enzyme lacking HNH nuclease activity through the binding sequence.

In certain embodiments, the complex further comprises: (c) a double-stranded target DNA sequence comprising a target strand and a complementary non-target strand; wherein the DNA-targeting sequence of the polynucleotide binds to the target strand; and, wherein the effector enzyme is capable of cleaving the non-target strand through the RuvC nuclease activity to release the 3′-end of the primer sequence, for priming reverse transcription by the reverse transcriptase, upon binding of the primer sequence to the complementary sequence to integrate the integrase or recombinase recognition sequence into the reverse transcription transcript.

In certain embodiments, the fusion protein further comprises a nuclear localization sequence (NLS).

In certain embodiments, the fusion protein further comprises a recombinase or an integrase.

In certain embodiments, the effector enzyme is a Cas9 nickase that lacks HNH endonuclease activity due to a point mutation at the endonuclease catalytic site of the HNH endonuclease.

In certain embodiments, the Cas9 nickase point mutation is H840A or equivalent thereof.

Another aspect of the invention provides a host cell comprising any one of the vector of the invention, or the plurality of vectors of the invention.

In certain embodiments, the vector further encodes the fusion protein of the invention.

In certain embodiments, the host cell further comprises a second vector encoding the fusion protein of the invention.

In certain embodiments, expression of the fusion protein is under the control of a constitutive promoter or an inducible promoter.

In certain embodiments, the host cell further comprises a donor sequence flanked by compatible integrase or recombinase recognition sequences that can direct the integration of the donor sequence into target DNA sequence comprising the integrase or recombinase recognition sequences, wherein the donor sequence is at least about 100 bp, 150 bp, 200 bp, 500 bp, 1 kb, 1.5 kb, 2, kb, 3 kb, 4 kb, 5 kb, 6 kb, 8 kb, or 10 kb.

In certain embodiments, the host cell is in a live animal.

In certain embodiments, the host cell is a cultured cell.

Another aspect of the invention provides a method of integrating a donor sequence into a target DNA sequence, the method comprising: (1) providing a complex of the invention (and the target DNA sequence if necessary), the integrase or recombinase (if necessary), and the donor sequence flanked by compatible integrase or recombinase recognition sequences; (2) allowing editing of the target DNA sequence to generate an edited target DNA sequence, by primer extension from the primer sequence, in order to insert integrase or recombinase recognition sequence according to the integrase or recombinase recognition sequence on the polynucleotide of the invention; and, (3) allowing insertion of the donor sequence into the edited target DNA sequence via site-specific recombination.

In certain embodiments, the complex is assembled inside a cell; wherein the target DNA sequence is a part of the genomic DNA of the cell; and wherein the polynucleotide of the invention, the vector of the invention, the fusion protein of the invention, or a polynucleotide encoding the fusion protein, and the donor sequence flanked by compatible integrase or recombinase recognition sequences are introduced into the cell.

In certain embodiments, the target DNA sequence is at, within, or adjacent to a target gene of interest (GOI).

In certain embodiments, the GOI is a defective gene, a disease gene, or a wild-type or mutant gene desired to be inactivated.

In certain embodiments, the target DNA sequence is within the first intron of the GOI.

Another aspect of the invention provides a kit comprising one or more of: (1) a polynucleotide of the invention, or a vector of the invention; (2) a second vector encoding a fusion protein of the invention, or the fusion protein of the invention formulated for delivery to a cell; optionally comprising in the same or different packaging a third vector encoding an integrase or a recombinase, or the integrase or recombinase formulated for delivery to the cell, and (3) a donor sequence flanked by integrase or recombinase recognition sequences.

In certain embodiments, the kit further comprises transformation, transfection, or infection reagents to facilitate the introduction of the vectors, fusion protein, or recombinase/integrase into the cell.

It should be understood that any embodiments described herein, including those only described in the Example section or only under one aspect of the invention, can be combined with any one or more other embodiments, unless specifically disclaimed or otherwise improper.

### BRIEF DESCRIPTION OF THE DRAWINGS

FIGS. 1A and 1B shows targeted insertion of donor DNA payload by combining prime editing and Bxb1 integrase-mediated recombination. FIG. 1A is a schematic drawing showing the insertion of a donor/payload sequence at a pre-determined target sequence (e.g., HEK3 locus). The donor/payload sequence was flanked by Bxb1 compatible AttB(gt) and AttB(ga) integrase recognition sequences. The orthogonal Bxb1 attP site pairs with GA and GT central dinucleotide were included in the template region of the prime editing guide RNA (pegRNA), which directs the incorporation of the AttP sequences into the HEK3 locus by the prime editing complex (PE2). Bxb1-mediated site-specific recombination between the AttP sites and the cognate AttB sites (flanking the payload sequence on a donor vector) resulted in donor/payload sequence insertion at the target locus. FIG. 1B shows the result of genotyping PCR using genomic target-specific primer (P1) and vector-specific primer (P2), which result revealed the presence of targeted insertion product in experimental sample (EXP) absent from control (CTL) sample which had received a pegRNA without the attP sites. Sequencing trace shows the correct junctional sequence derived from the genomic target and donor vector as well as the recombined AttR (AttP×AttB) site.

FIGS. 2A and 2B shows targeted insertion of donor DNA payload by combining prime editing and dual integrase-mediated recombination. FIG. 2A is a schematic drawing showing the insertion of a donor/payload sequence at a pre-determined target sequence (e.g., HEK3 locus). The donor/payload sequence was flanked by Bxb1 compatible AttB(Bxb1) and PhiC31 compatible AttB(PhiC31) integrase recognition sequences. The orthogonal Bxb1 attP site and the PhiC31 attP site were included in the template region of the prime editing guide RNA (pegRNA), which directs the incorporation of the AttP sequences into the HEK3 locus by the prime editing complex (PE2). Bxb1- and PhiC31-mediated site-specific recombination between the AttP sites and the cognate AttB sites (flanking the payload sequence on a donor vector) resulted in donor/payload sequence insertion at the target locus. FIG. 2B shows the result of genotyping PCR using genomic target-specific primer (P1) and vector-specific primer (P2), which result revealed the presence of targeted insertion product in experimental sample (EXP) absent from control (CTL) sample which had received a pegRNA without the attP sites. Sequencing trace shows the precise junctional sequences derived from the genomic target and donor vector and the recombined Bxb1 AttR (AttP×AttB) site.

FIGS. 3A and 3B shows targeted insertion of donor DNA payload by combining prime editing and FlpE recombinase-mediated recombination. FIG. 3A is a schematic drawing showing the insertion of a donor/payload sequence at a pre-determined target sequence (e.g., HEK3 locus). The donor/payload sequence was flanked by a pair of FRT sequences in the same orientation. A single copy of the same FRT sequence was included in the template region of the prime editing guide RNA (pegRNA), which directs the incorporation of the FRT sequence into the HEK3 locus by the prime editing complex (PE2). Flipase-mediated site-specific recombination between the FRT sites resulted in donor/payload sequence insertion at the target locus. FIG. 3B shows the result of sequencing trace, including the precise junctional sequences derived from the genomic target and donor vector and the reconstituted FRT site.

### DETAILED DESCRIPTION OF THE INVENTION

### 1. Overview

The invention described herein, also known as Primas (Prime editing, Recombinase, Integrase-mediated Addition of Sequence), provides methods and reagents for site-specifically or sequence-specifically inserting large polynucleotide fragments (e.g., longer than 45 bp, 50 bp, 100 bp, 200 bp, 1 kb, 2 kb, 3, kb, 5 kb, 10 kb, 20 kb, 50 kb or more) into a target double-stranded DNA sequence, such as a genomic DNA sequence.

The invention described herein takes advantage of certain site-specific recombinases, integrases, or transposases (or “recombinase/integrase” or simply “recombinase” for convenience of description) to insert large DNA fragments into a target DNA sequence, which target DNA sequence has previously been modified via prime editing-mediated insertion to incorporate one or more recognition sequence(s) for the site-specific recombinases, integrases, or transposases.

Site-specific recombinases and integrases (e.g., Bxb1, PhiC31, Cre, FlpE, Tn7) can mediate insertion of DNA via specific recognition sequences specific for the recombinases and integrases. According to the invention, the recombinase/integrase/transposase recognition sites are encoded on a pegRNA and inserted into the target site via prime editing. A recombinase/integrase/transposase uses the inserted recognition site at the target locus to incorporate a donor DNA flanked by cognite recombinase/integrase/transposase sites.

A salient feature of the present invention is the presence of a site-specific integrase or recombinase recognition sequence or complement thereof within the template region of the pegRNA, i.e., the region of the pegRNA that encodes heterologous sequences to be inserted into the target DNA sequence.

Thus in one aspect, the invention provides a polynucleotide comprising, not necessarily in this order: (1) a DNA-targeting sequence that is complementary to the target strand (TS) of a double-stranded target DNA sequence, the target strand is complementary to the non-target strand (NTS) of the double-stranded target DNA sequence; (2) a binding sequence for a CRISPR-Cas system effector enzyme (such as a Type II or Type V Class II CRISPR-Cas system effector enzyme); (3) an integrase or recombinase or transposase recognition sequence or complement thereof; and, (4) a primer binding sequence complementary to a primer sequence, wherein optionally, the primer sequence comprises the 3′ end resulting from the cleavage of the non-target strand by the (e.g., RuvC or RuvC-like nuclease activity of) the Cas effector enzyme, when the polynucleotide is complexed with the effector enzyme via the binding sequence and guides the cleavage of the non-target strand by the RuvC nuclease activity of the effector enzyme.

In an alternative aspect, the invention provides a pegRNA (primer editing guide RNA) comprising a recombinase or integrase or transposase recognition sequence (or complement thereof), wherein the recombinase or integrase recognition sequence (or complement thereof) is positioned on the pegRNA for insertion into a target DNA of the pegRNA. Preferably, the recombinase or integrase or transposase recognition sequence (or complement thereof) is in the template region of the pegRNA, (e.g., is upstream of the primer binding sequence in certain configurations), such that when the primer binding sequence is paired with the primer sequence resulting from cleavage by the CRISPR-Cas system effector enzyme, the recombinase or integrase or transposase recognition sequence (or complement thereof) is inserted into the target DNA cleavage site.

Another aspect of the invention provides a vector encoding the polynucleotide of the invention.

Another aspect of the invention provides a complex comprising: (a) any one of the polynucleotide of the invention, and, (b) a fusion protein comprising: (i) a CRISPR-Cas system effector enzyme (such as a Type II or Type V Class II CRISPR-Cas system effector enzyme) lacking HNH nuclease activity or an equivalent nuclease activity; and, (ii) a reverse transcriptase; wherein the polynucleotide is complexed with the effector enzyme lacking HNH or equivalent nuclease activity through the binding sequence. In certain embodiment, the fusion protein further comprises (iii) a recombinase or integrase or transposase.

Another aspect of the invention provides a complex comprising: (a) any one of the polynucleotide of the invention, and, (b) a CRISPR-Cas system effector enzyme (such as a Type II or Type V Class II CRISPR-Cas system effector enzyme) lacking HNH or equivalent nuclease activity; wherein the polynucleotide of the invention further comprises a recruitment sequence for a reverse transcriptase, a recombinase, an integrase or a transposase; wherein the polynucleotide (i) is complexed with the effector enzyme lacking HNH or equivalent nuclease activity through the binding sequence, and (ii) is complexed with the reverse transcriptase, recombinase, integrase or transposase through the recruitment sequence.

The recruitment sequence can be any nucleotide sequence motifs capable of being bound by a protein domain fused to the reverse transcriptase, a recombinase, an integrase or a transposase. For example, the protein domain/recruitment sequence pair can be any one of: MS2 bacteriophage coat protein/a stem-loop structure from the MS2 phage genome (MS2 binding site or MBS); PP7 bacteriophage coat protein (PCP)/PP7 binding site (PBS); or lambdaN Peptide/its specific 19 nt binding site (boxB). Additional protein domain/recruitment sequence pair can include any of the PUF domain/PUF recognition sequences, such as those disclosed in WO2016/148994 (incorporated herein by reference).

Another aspect of the invention provides a complex comprising: (a) any one of the polynucleotide of the invention, and, (b) a CRISPR-Cas system effector enzyme (such as a Type II or Type V Class II CRISPR-Cas system effector enzyme) lacking HNH or equivalent nuclease activity; wherein the complex further comprises one or more of (1) a reverse transcriptase; and (2) a recombinase, an integrase or a transposase; wherein (1) or (2) or both is/are either fused to the Cas effector enzyme as a fusion protein, or is/are recruited to the polynucleotide of the invention through protein domain/recruitment sequence pair (wherein the protein domain is fused to (1) and/or (2), and wherein the recruitment sequence is in the polynucleotide of the invention). In this configuration, the polynucleotide of the invention (i) is complexed with the effector enzyme lacking HNH or equivalent nuclease activity through the binding sequence, and (ii) is complexed with the reverse transcriptase, recombinase, integrase or transposase through the recruitment sequence, if the reverse transcriptase, recombinase, integrase or transposase is not already fused with the Cas effector enzyme.

In certain embodiments, the complex further comprises (c) a double-stranded target DNA sequence comprising a target strand and a complementary non-target strand; wherein the DNA-targeting sequence of the polynucleotide binds to the target strand; and, wherein the effector enzyme is capable of cleaving the non-target strand through the RuvC or RuvC-like nuclease activity to release the 3′-end of the primer sequence, for priming reverse transcription by the reverse transcriptase, upon binding of the primer sequence to the complementary sequence to integrate the integrase or recombinase recognition sequence into the reverse transcription transcript.

Another aspect of the invention provides a host cell comprising any one of the vector of the invention, or any one of the plurality of vectors of the invention.

Another aspect of the invention provides a method of integrating a donor sequence into a target DNA sequence, the method comprising: (1) providing a complex of the invention (and the target DNA sequence if necessary), the reverse transcriptase, integrase, recombinase, and/or transposase (if necessary), and the donor sequence flanked by compatible integrase or recombinase or transposase recognition sequences; (2) allowing editing of the target DNA sequence to generate an edited target DNA sequence, by primer extension from the primer sequence, in order to insert integrase or recombinase or transposase recognition sequence according to the integrase or recombinase or transposase recognition sequence on the polynucleotide of the invention; and, (3) allowing insertion of the donor sequence into the edited target DNA sequence via site-specific recombination.

Another aspect of the invention provides a kit comprising one or more of: (1) a polynucleotide of the invention, or a vector of the invention; (2) a second vector encoding a Cas effector enzyme or fusion protein of the invention, or the Cas effector enzyme/fusion protein of the invention formulated for delivery to a cell; optionally comprising in the same or different packaging a third vector encoding an integrase or a recombinase or transposase, or the integrase or recombinase or transposase formulated for delivery to the cell, and (3) a donor sequence flanked by integrase or recombinase or transposase recognition sequences.

With the invention generally described above, various features of the invention will be further elaborated below. It should be understood that features of the invention, even when described in the context of separate embodiments, or even separate embodiments under different aspects of the invention, may be provided in combination in a single embodiment. Conversely, various features of the invention described in the context of a single embodiment, may also be provided separately or in any suitable subcombination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### 2. The Polynucleotide of the Invention

The polynucleotide of the invention comprises four sequence segments, not necessarily in any particular order listed herein: i) a first segment comprising a nucleotide sequence (i.e., DNA-targeting sequence, or spacer sequence) that is complementary to a target sequence, more precisely the target strand (TS) of the target DNA sequence; ii) a second segment (e.g., the binding sequence) that interacts with/bind to/forms a complex with a CRISPR-Cas system effector enzyme (such as a Type II or Type V Class II CRISPR-Cas system effector enzyme), such as Cas9 or Cpf1 (e.g., wt or nickase, such as a nickase with RuvC-like nuclease activity); iii) one or more copies of an integrase or recombinase or transposase recognition sequence or complement thereof; and iv) a primer binding sequence complementary to a primer sequence, wherein the primer sequence comprises the 3′ end resulting from the cleavage of the non-target strand of the target DNA sequence by the RuvC-like nuclease activity of the effector enzyme, when the polynucleotide is complexed with the effector enzyme via the binding sequence and guides the cleavage of the non-target strand by the RuvC-like nuclease activity of the effector enzyme.

The four segments may be linked together through one or more optional linker sequences. In certain embodiments, any two adjacent segments may be linked directly, without any linker sequence. In other embodiments, any two adjacent segments may be linked to each other through a linker sequence.

In some embodiments, the polynucleotide of the invention comprises additional segements such as one or more recruitment sequence for recruiting one or more of reverse transcriptase, recombinase, integrase, and/or transposase which has been fused to a protein domain that binds to the recruitment sequence.

In a related aspect, the polynucleotide of the invention is a pegRNA (primer editing guide RNA) comprising a recombinase or integrase or transposase recognition sequence (or complement thereof), wherein the recombinase or integrase or transposase recognition sequence (or complement thereof) is positioned on the pegRNA for insertion into a target DNA of the pegRNA.

In certain embodiments, the polynucleotide is an RNA. The RNA can be transcribed from a vector encoding the RNA, using a constitutive or inducible promoter, such as U6 promoter.

In certain other embodiments, the polynucleotide may comprise modified or non-natural nucleotides to, for example, enhance stability, binding affinity, and/or to avoid unwanted folding. More than 100 different base modifications in RNA are known (such as methylations and uridine isomerization), each of which may perform a plethora of functions. Such modified RNA many be synthetic and can be directly delivered to a target cell by art-recognized means such as transfection or nanoparticle-mediated delivery. For example, the nanoparticles can comprise polymers, such as cationic polymers. Synthetic polymers such as poly-L-lysine, polyamidoamine, and polyethyleneimine, Poly(β-amino esters), as well as naturally occurring polymers such as chitosan, can be used for RNA delivery. Lipids and lipid-like materials, including cationic lipids and self-assembly lipids known as lipoplexes, are another major class of nanoparticle-based delivery vehicles for RNA. Such lipid based nanoparticles (LNP) may optionally comprise other hydrophobic moieties, such as cholesterol and PEG-lipid, in addition to an ionizable/cationic lipid, to enhance nanoparticle stability and/or efficacy of RNA delivery. The RNA can be modified by means of chemical alterations to the ribose sugar (of particular importance at the 2′ position), the phosphate linkage, and/or the individual bases. Exemplary modifications include: pseudouridine, 5-Bromo-uridine, 5-methylcytidine, 2′-deoxy, 2′-OMe, amide linkage, and thioate linkage.

The target sequence is a double-stranded DNA, such as a genomic DNA. The first segment comprises a nucleotide sequence complementary to the target strand (TS) of the target DNA sequence. The other strand of the target DNA sequence is the non-target strand (NTS).

In certain embodiments, the four segments i)-iv) are arranged, in that order, from 5′ to 3′. In this embodiment, the DNA-targeting sequence is the most 5′ end of the subject polynucleotide, followed by a more 3′ binding sequence for forming a complex with the Cas effector enzyme, which binding sequence extends further 3′ into the one or more copies of the recombinase/integrase recognition sequence(s), which is followed by the most 3′ of the four segments—the primer binding sequence. An exemplary polynucleotide of the invention is compatible with Cas9 or related Cas effector enzymes, which utilize a single guide RNA (sgRNA) sequence having a tracrRNA covalently linked to the 3′ end of the crRNA.

In certain embodiments, the four segments i)-iv) are arranged, from 5′ to 3′, iv)-iii)-i)-ii). In this embodiment, the DNA-targeting sequence and the binding sequence have the same order or orientation as in the previous embodiment, which is compatible with Cas9 and related effector enzymes. The primer binding sequence, however, is further 5′ to the DNA-targeting sequence, and anneal to the primer sequence comprising the 3′ end resulting from the cleavage of the non-target strand by the RuvC nuclease activity of the effector enzyme (e.g., Cas9). Reverse transcription from the newly generated 3′ end of the primer sequence can produce a cDNA, using a template comprising the one or more integrase/recombinase recognition sequence or complement thereof that is situated at the most 5′ end of the subject polynucleotide. In certain embodiments, the NTS is nicked by a nuclease.

In certain embodiments, the four segments i)-iv) are arranged, from 5′ to 3′, iv)-iii)-ii)-i). In this embodiment, the DNA-targeting sequence is the most 3′ of the segments, and the binding sequence is immediately 5′ to the DNA-targeting sequence. This configuration is compatible with Cpf1 and the related Type V Class II Cas effector enzymes. The primer binding sequence is further 5′ to the binding sequence, and anneal to the primer sequence comprising the 3′ end resulting from the cleavage of the non-target strand by the RuvC nuclease activity of the effector enzyme (e.g., Cpf1). Reverse transcription from the newly generated 3′ end of the primer sequence can produce a cDNA, using a template comprising the one or more integrase/recombinase recognition sequence or complement thereof that is situated at the most 5′ end of the subject polynucleotide.

In certain embodiments, the four segments i)-iv) are arranged, from 5′ to 3′, ii)-i)-iii)-iv). In this embodiment, the DNA-targeting sequence is also 3′ to the most 5′ end of the segments—the binding sequence. The DNA-targeting sequence is followed by the more 3′ one or more copies of the recombinase/integrase recognition sequence(s), which is followed by the most 3′ of the four segments—the primer binding sequence. This configuration is also compatible with Cpf1 and effector enzymes with the binding sequence 5′ to the DNA-targeting sequence.

Additional features of the different segments are further described below.

a. DNA-Targeting Sequence

The DNA-targeting sequence is functionally similar or equivalent to the crRNA or guide RNA or gRNA of the CRISPR/Cas complex/system. However, in the context of the instant invention, the DNA-targeting sequence may not originate from any particular crRNA or gRNA, but can be arbitrarily designed based on the sequence of the target polynucleotide sequence.

The DNA-targeting sequence comprises a nucleotide sequence that is complementary to a specific sequence within a target DNA (or more precisely the TS of the double-stranded target DNA). In other words, the DNA-targeting sequence interacts with a target strand (TS) sequence of the target DNA in a sequence-specific manner via annealing or hybridization (i.e., base pairing). As such, the nucleotide sequence of the DNA-targeting sequence may vary, and it determines the location within the target DNA that the subject polynucleotide and the target DNA will interact. The DNA-targeting sequence can be modified or designed (e.g., by genetic engineering) to hybridize to any desired sequence within the double-stranded target DNA.

The DNA targeting sequence is also designed such that it is capable of guiding the RuvC nuclease of the Cas effector enzyme to cleave the NTS of the target DNA, based on the proper spacing with a PAM (protospacer adjacent motif) sequence adjacent to the desired cleavage site. The PAM sequence is generally specific for the Cas effector enzyme to be used with the polynucleotide of the invention.

For example, in certain embodiments, when the Cas effector enzyme is Cas9, the target strand sequence is immediately 3′ to a PAM sequence of the complementary strand, which can be 5′-CCN-3′, wherein N is any DNA nucleotide. That is, in this embodiment, the complementary strand (NTS) of the target strand (TS) polynucleotide sequence is immediately 5′ to a PAM sequence that is 5′-NGG-3′, wherein N is any DNA nucleotide. In related embodiments, the PAM sequence of the complementary strand (NTS) matches the wt Cas9. See above for the PAM sequences from species other than S. pyogenes.

In other embodiments, different Cas effector enzymes have different sequences and different relative locations of the respective PAM sequences. For example, Cpf1 has a PAM sequence of 5′ TTN 3′ on the NTS that is 5′ to the TS sequence bound by the DNA-binding sequence. Other PAM sequences for the other Type II and V Class 2 Cas effectors are known in the art, and are incorporated herein by reference.

In certain embodiments, the DNA-binding sequence of the subject polynucleotide is designed to match (either perfectly or with some permissible degrees of mismatch, see below) the TS of a target double-stranded DNA (such as a genomic DNA) sequence, within the proper context of an adjacent PAM sequence compatible with the Cas effector enzyme to be used with the subject polynucleotide (e.g., the Cas effector enzyme that can bind to the binding sequence).

The DNA-targeting sequence can have a length of from about 12 nucleotides to about 100 nucleotides. For example, the DNA-targeting sequence can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the DNA-targeting sequence can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

In certain embodiments, the DNA-targeting sequence is about 11-13 bases in length, about 14-20 bases in length, about 14-25 bases in length, about 19-22 bases in length, about 21-72 bases in length, or about 32-38 bases in length.

The nucleotide sequence of the DNA-targeting sequence that is complementary to a target strand polynucleotide sequence of the target DNA can have a length of at least about 12 nt. For example, the DNA-targeting sequence that is complementary to a target polynucleotide sequence of the target DNA can have a length at least about 12 nt, at least about 15 nt, at least about 18 nt, at least about 19 nt, at least about 20 nt, at least about 25 nt, at least about 30 nt, at least about 35 nt or at least about 40 nt. For example, the DNA-targeting sequence that is complementary to a target strand sequence of a target DNA can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. The nucleotide sequence of the DNA-targeting sequence that is complementary to the target strand sequence of the target DNA can have a length of at least about 12 nt.

In certain embodiments, the DNA-targeting sequence is complementary to the target strand of the double-stranded target DNA sequence over about 12-22 nucleotides (nts), about 14-20 nts, about 16-20 nts, about 18-20 nts, or about 12, 14, 16, 18, or 20 nts (preferably, the complementary region comprises a continuous stretch of 12-22 nts, preferably at the 3′ end of the DNA-binding sequence).

In some cases, the DNA-targeting sequence that is complementary to a target strand sequence of the target DNA is 20 nucleotides in length. In some cases, the DNA-targeting sequence that is complementary to a target polynucleotide sequence of the target DNA is 19 nucleotides in length.

In certain embodiments, the DNA-targeting sequence is compatible with the Cas effector enzyme that can bind to the binding sequence of the subject polynucleotide.

In certain embodiments, the Cas effector enzyme is Cas9, and the DNA-targeting sequence is 19-22 nt, 19-21 nt, 20-22 nt, 20-21 nt, or about 20 nt.

In certain embodiments, the Cas effector enzyme is Cpf1, and the DNA-targeting sequence is 20-24 nt, 21-23 nt, 21-22 nt, or about 21 nt.

The percent complementarity between the DNA-targeting sequence and the target strand sequence of the target DNA can be at least 50% (e.g., at least 55%, at least 60%, at least 65%, at least 70%, at least 75%, at least 80%, at least 85%, at least 90%, at least 95%, at least 97%, at least 98%, at least 99%, or 100%). In some cases, the percent complementarity between the DNA-targeting sequence and the target strand sequence is 100% over the seven or eight contiguous 5′-most nucleotides of the target polynucleotide sequence. In some cases, the percent complementarity between the DNA-targeting sequence and the target strand sequence is at least 60% over about 20 contiguous nucleotides. In some cases, the percent complementarity between the DNA-targeting sequence and the target strand sequence is 100% over the 7, 8, 9, 10, 11, 12, 13, or 14 contiguous 5′-most nucleotides of the target polynucleotide sequence (i.e., the 7, 8, 9, 10, 11, 12, 13, or 14 contiguous 3′-most nucleotides of the DNA-targeting sequence), and as low as 0% over the remainder. In such a case, the DNA-targeting sequence can be considered to be 7, 8, 9, 10, 11, 12, 13, or 14 nucleotides in length, respectively.

In certain embodiments, the DNA-targeting sequence is at least about 60%, 70%, 80%, 85%, 90%, 95% or more complementary to the target strand.

b. Binding Sequence

The Cas effector protein-binding segment or Cas effector protein-binding sequence (or simply “binding sequence”) of the subject polynucleotide binds to a wild-type (wt) Cas effector enzyme (such as wt Cas9 or Cpf1), or a modified Cas protein thereof (e.g., nickase) with reduced endonuclease activity, or lacks endonuclease activity in one of the two endonucleases of the effector enzyme, such as a nickase with RuvC or RuvC like activity that cleaves the NTS. For simplicity, the protein-binding sequence of the subject polynucleotide, which may bind to wt and/or modified Cas effector enzymes, may simply be referred to as “Cas-binding sequence” herein. However, it should be understood that when the Cas-binding sequence of the invention binds to a nickase, it is not prevented from binding to a wt Cas effector enzyme. In certain embodiments, the Cas-binding sequence of the invention binds to wt as well as nickase.

The Cas-binding sequence interacts with or bind to a Cas effector enzyme (e.g., wt or nickase), and together they bind to the target polynucleotide sequence recognized by the DNA-targeting sequence.

In Cas9 like proteins, the Cas-binding sequence may comprise two complementary stretches of nucleotides that hybridize to one another to form a double stranded RNA duplex (a dsRNA duplex). These two complementary stretches of nucleotides may be covalently linked by intervening nucleotides known as linkers or linker nucleotides (e.g., in the case of a single-molecule polynucleotide), and hybridize to form the double stranded RNA duplex (dsRNA duplex, or “Cas9-binding hairpin”) of the Cas9-binding sequence, thus resulting in a stem-loop structure. Alternatively, in some embodiment, the two complementary stretches of nucleotides may not be covalently linked, but instead are held together by hybridization between complementary sequences (e.g., in the case of a two-molecule polynucleotide of the invention).

The binding sequences are specific for the Cas effector enzymes, and are known in the art (incorporated herein by reference).

In certain embodiments, the Cas-binding sequence (such as Cpf1- or Cas9-binding sequence) can have a length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the Cas-binding sequence (such as Cpf1- or Cas9-binding sequence) can have a length of from about 15 nucleotides (nt) to about 80 nt, from about 15 nt to about 50 nt, from about 15 nt to about 40 nt, from about 15 nt to about 30 nt, from about 37 nt to about 47 nt (e.g., 42 nt), or from about 15 nt to about 25 nt.

In certain embodiments, the binding sequence is about 20-25 nt, about 37-47 nt, or about 42 nt.

The dsRNA duplex of the Cas-binding sequence (such as Cpf1- or Cas9-binding sequence) can have a length from about 6 base pairs (bp) to about 50 bp. For example, the dsRNA duplex of the Cas9-binding sequence can have a length from about 6 bp to about 40 bp, from about 6 bp to about 30 bp, from about 6 bp to about 25 bp, from about 6 bp to about 20 bp, from about 6 bp to about 15 bp, from about 8 bp to about 40 bp, from about 8 bp to about 30 bp, from about 8 bp to about 25 bp, from about 8 bp to about 20 bp or from about 8 bp to about 15 bp. For example, the dsRNA duplex of the Cas9-binding sequence can have a length from about from about 8 bp to about 10 bp, from about 10 bp to about 15 bp, from about 15 bp to about 18 bp, from about 18 bp to about 20 bp, from about 20 bp to about 25 bp, from about 25 bp to about 30 bp, from about 30 bp to about 35 bp, from about 35 bp to about 40 bp, or from about 40 bp to about 50 bp. In some embodiments, the dsRNA duplex of the Cas-binding sequence (such as Cas9-binding sequence) has a length of 36 base pairs. In some embodiments, the dsRNA duplex of the Cas-binding sequence (such as Cpf1-binding sequence) has a length of about 20-24 base pairs. The percent complementarity between the nucleotide sequences that hybridize to form the dsRNA duplex of the Cas-binding sequence can be at least about 60%. For example, the percent complementarity between the nucleotide sequences that hybridize to form the dsRNA duplex of the Cas-binding sequence can be at least about 65%, at least about 70%, at least about 75%, at least about 80%, at least about 85%, at least about 90%, at least about 95%, at least about 98%, or at least about 99%. In some cases, the percent complementarity between the nucleotide sequences that hybridize to form the dsRNA duplex of the Cas-binding sequence is 100%.

The linker can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker is 4 nt.

Non-limiting examples of nucleotide sequences that can be included in a suitable Cas9-binding sequence (i.e., Cas9 handle) are set forth in SEQ ID NOs: 563-682 of WO 2013/176772 (see, for examples, FIGS. 8 and 9 of WO 2013/176772), incorporated herein by reference.

In some cases, a suitable Cas-binding sequence (such as Cpf1- or Cas9-binding sequence) comprises a nucleotide sequence that differs by 1, 2, 3, 4, or 5 nucleotides from any one of the above-listed sequences.

c. Integrase/Recombinase/Transposase and Recognition Sequences Thereof

The integrase, recombinase, and/or transposase of the invention (or “recombinase” for short) facilitate site-specific recombination to insert a donor sequence into a target double-stranded DNA (or target DNA).

“Site-specific recombination,” as used herein, includes genetic recombination in which DNA strand exchange takes place between segments possessing a certain degree of sequence homology, catalyzed by site-specific recombinases (SSRs). SSRs perform rearrangements of DNA segments by recognizing and binding to short, specific DNA sequences (SSR recognition sequences or simply “SSR sites”), at which they cleave the DNA backbone, exchange the two DNA helices involved, and rejoin the DNA strands.

“Site-specific recombinase (SSR)” (or “recombinase” for short) as used herein include recombinase, integrase, or transposase, such as those described in more detail herein below.

In certain embodiments, the recombinase is sufficient to facilitate SSR by itself at the presence of a compatible recombination site.

In certain embodiments, the recombinase is itself insufficient to facilitate SSR at the presence of a compatible recombination site, and requires the presence of one or more accessory proteins and/or accessory sites.

In certain embodiments, the recombination site is between 30 and 200 nucleotides in length, and consists of two motifs with a partial inverted-repeat symmetry, to which the recombinase binds, and which flank a central crossover sequence for the recombination.

In certain embodiments, the pairs of sites between which the recombination occurs are identical. In certain embodiments, the pairs of sites between which the recombination occurs are different (e.g., attP and attB of λ integrase).

In certain embodiments, the integrase or recombinase recognition sequence comprises only one recognition sequence (such as FRT or attTn7) for targeted insertion utilized by certain recombinases/transposases.

In certain embodiments, the integrase or recombinase recognition sequence comprises two or more recognition sequences. The two or more recognition sequences can be identical or different (such as two orthogonal sites (e.g., the Bxb1-ga,gt sites in the examples)), and/or can be in the same or opposite orientation. In certain embodiments, the recognition sequences may be for two different integrases or recombinases.

In certain embodiments, the integrase or recombinase recognition sequence comprises three or more sites to allow a mixture of recombinase/integrase donors, or to allow generation of cells harboring different insertions/orientation of insertions.

In certain embodiments, the recombinase is a tyrosine (Tyr) recombinase (subfamily A1 recombinase), such as a Cre recombinase (e.g., from the P1 phage), or a FLP (e.g., from Saccharomyces cerevisiae). Other Tyr recombinase include: Dre, KD, B2 and B3. The recognition sequences for these Tyr recombinases are known in the art, selected few of which are described in more details below. In general, Tyr recombinase recognition sequences are generally 34/48 bps.

In certain embodiments, the recombinase is a Tyr integrase (subfamily A2 recombinase), such as a bacterial lambda phage integrase (which utilizes attP/attB recognition sites). Additional Tyr integrases include HK022 and HP1. These Tyr integrases may require accessory factors such as Xis and IHF.

In certain embodiments, the recombinase is a serine (Ser) recombinase (subfamily B1 recombinase), such as a Ser resolvase or a DNA invertase. Examples of such recombinases include the gamma-delta resolvase (e.g., from the Tn1000 transposon), a Tn3 resolvase (from the Tn3 transposon), ParA and Gin. Such Ser resolvase/invertases generally do not require additional accessory factors to function, and they recognize attP/attB sites.

In certain embodiments, the recombinase is a serine (Ser) integrase (subfamily B2 recombinase), such as φC31 (from the φC31 phage), Bxb1, and R4. Such Ser integrases may require additional accessory factors such as RDF to function, and they recognize attP/attB sites.

The recombinase of the invention recombines two target sites, which are either identical (subfamily A1) or distinct (phage-derived enzymes in subfamilies A2, B1 and B2). For subfamily A1 recombinases, the recognition sites have individual designations (such as “FRT” for Flp-recombinase, “loxP” for Cre-recombinase). Meanwhile, “attP” and “attB” (attachment sites on the phage and bacterial part, respectively) are used for the other recombinase subfamilies. Subfamily A1 recombinases have short (usually 34 bp-) sites consisting of two (near-)identical 13 bp arms (arrows) flanking an 8 bp spacer (the crossover region). For Flp, there is an alternative, 48 bp site available with three arms, each accommodating a Flp unit (a so-called “promoter”). The attP- and attB-sites follow similar architectural rules, but the arms show only partial identity and differ in both cases. As a result, recombination of two identical sites leads to reversible conversion product sites with the same composition, although they contain arms from both substrates. Meanwhile, in case of attP×attB recombination, crossovers can only occur between these complementary partners in processes that lead to two different end products in an irreversible fashion (attP×attB→attR+attL).

Thus, in certain embodiments, the reaction catalyzed by the recombinase is reversible.

In other embodiments, the reaction catalyzed by the recombinase is irreversible.

Specific exemplary recombinases that can be suitable for use in the invention are provided below.

### Cre-loxP

In certain embodiments, the recombinase is a Cre recombinase, which is a member of the integrase family of site specific recombinase, and requires no additional cofactors (such as ATP) or accessory proteins for its function.

Cre catalyzes site specific recombination between two DNA recognition sites known as LoxP sites, which are 34 bp sequences consisting of two 13 bp palindromic sequences that flank an 8 bp spacer region which is variable except for the middle two bases. The exact sequence of loxP is:

### 13 bp 8 bp 13 bp

### ATAACTTCGTATA-NNNTANNN-TATACGAAGTTAT

“N” indicates bases which may vary. Since the 13 bp sequences are palindromic but the 8 bp spacer is not, the loxP sequence has a certain direction. The Cre-loxP system can be used to carry out deletions, insertions, translocations and inversions at specific sites in the target DNA. The products of Cre-mediated recombination at loxP sites depend on the location and relative orientation of the loxP sites. In certain embodiments, the two loxP sites are in the same orientation, and the floxed sequence (i.e., sequence flanked by two loxP sites) is excised. In certain embodiments, the two loxP sites are in the opposite orientation, and the floxed sequence is inverted. In certain embodiments, a donor sequence is floxed, and the donor sequence is swapped with the original sequence flanked by loxP sites inserted into the target DNA by the method and polkynucleotide of the invention.

In certain embodiments, mutated loxP sites are used as recombinase recognition sequences to confer one or more advantages, including reducing reversible reaction, preventing unintended excision in trans (instead of an in cis cassette exchange event).

Example Alternate loxP Sites[21]

| | 13 bp | 8 bp | 13 bp |
|---|---|---|---|
| | Recognition | Spacer | Recognition |
| Name | Region | Region | Region |
| Wild-Type | ATAACTTCGTATA | ATGTATGC | TATACGAAGTTAT |
| lox 511 | ATAACTTCGTATA | ATGTATaC | TATACGAAGTTAT |
| lox 5171 | ATAACTTCGTATA | ATGTgTaC | TATACGAAGTTAT |
| lox 2272 | ATAACTTCGTATA | AaGTATcC | TATACGAAGTTAT |
| M2 | ATAACTTCGTATA | AgaaAcca | TATACGAAGTTAT |
| M3 | ATAACTTCGTATA | taaTACCA | TATACGAAGTTAT |
| M7 | ATAACTTCGTATA | AgaTAGAA | TATACGAAGTTAT |
| M11 | ATAACTTCGTATA | cgaTAcca | TATACGAAGTTAT |
| lox 71 | TACCGTTCGTATA | NNNTANNN | TATACGAAGTTAT |
| lox 66 | ATAACTTCGTATA | NNNTANNN | TATACGAACGGTA |

* lowercase letters indicate bases that have been mutated from the wild-type.

Thus, in certain embodiments, the polynucleotide and system/method of the invention can be used to insert two loxP sites in a site-specific manner, to either excise or reverse the DNA sequence between the two inserted loxP sites. For example, the excision can be used to delete a DNA fragment that may harbor a disease gene (such as one that produces a dominant negative disease protein). Meanwhile, in certain embodiments, the reversion of a DNA fragment may be used to “cure” an inversion mutation in the genomic sequence.

A variant of the Cre recombinase is Tre recombinase, which is an engineered Cre that removes DNA inserted by HIV from infected cells. It was generated through selective mutation of the Cre recombinase such that it recognizes HIV long terminal repeats (loxLTR). As a result, instead of performing Cre-Lox recombination, Tre recombinase performs recombination at HIV provirus sites. The Tre-loxLTR system can be used in place of Cre-loxP in the system and method of the invention.

### Flp-FRT

In certain embodiments, the recombinase is a tyrosine family site-specific recombinase flippase (Flp), and the recognition sequence is flippase recognition target (FRT) sites. The Flp-FRT recombination is analogous to Cre-lox recombination. The FRT sites and the flippase (Flp) are derived from the 2μ plasmid of Saccharomyces cerevisiae. The 34 bp minimal FRT site sequence is:

5′ GAAGTTCCTATTCtctagaaaGtATAGGAACTTC 3′

The flippase (Flp) binds to both 13-bp 5′-GAAGTTCCTATTC-3′ arms flanking the 8 bp spacer in reverse orientation. FRT-mediated cleavage occurs just ahead from the asymmetric 8 bp core region (5′ tctagaaa 3′) on the top strand and behind this sequence on the bottom strand. Several variant FRT sites exist, but recombination can usually occur only between two identical FRTs but generally not among non-identical (or “heterospecific”) FRTs. Thus in certain embodiments, different pairs of FRT sequences can be inserted to the target DNA to insert different donor sequences flanked by corresponding matching FRT sequences.

Specifically, most base substitutions (such as G to A, T, and C, etc.) in only one of the two FRT sites only cause minimal effects. However, when mutations occur within both FRT sites, the efficiency of FLP is dramatically reduced. In addition, nucleotides that are most crucial for the binding of FLP and efficacy of the site-specific recombination have been identified, including the capitalized first, the third, and the seventh nucleotide of 5′-GaAtagGaacttc-3′.

In certain embodiments, the FRT sequence is modified to include an additional arm sequences (5′-GAAGTTCCTATTCC-3′) one base pair away from the upstream element and in the same orientation, as in 5′-GAAGTTCCTATTCcGAAGTTCCTATTCtctagaaaGtATAGG AACTTC-3′. This segment is dispensable for excision but essential for integration, including Recombinase-mediated cassette exchange.

Like the Cre-loxP system, Flp-FRT system can be used to carry out deletions (excisions), insertions, translocations and inversions at specific sites in the target DNA. The products of Flp-FRT-mediated recombination at FRT sites depend on the location and relative orientation of the FRT sites. In certain embodiments, two FRT sites flanking a donor sequence are in the same orientation, and the donor sequence is inserted into a single matching FRT site on the target DNA. In certain embodiments, two FRT sites flanking a target sequence to be deleted on the target DNA (inserted by the method and polynucleotides of the invention) are in the same orientation, and the target sequence is deleted from the target DNA in the presence of a single matching FRT site on, for example, a separate DNA construct, such as a plasmid. In certain embodiments, two FRT sites are inserted into and flanks the target DNA sequence, in opposite orientation, and the target DNA flanked by the FRT sequences is inverted. In certain embodiments, a first FRT sequence is inserted into a first target DNA, and a second matching FRT sequence is inserted into a second target DNA, and the two target DNAs are fused at one FRT site via translocation.

### PhiC31 and Bxb1 Integrases

In certain embodiments, the recombinase is a PhiC31 Ser integrase. Contrary to the other Tyr recombinases, PhiC31-INT acts in a unidirectional manner, firmly locking in the donor vector at a genomically anchored target. An obvious advantage of this system is that it can rely on unmodified, native attP (acceptor) and attB donor sites. Additional benefits may arise from the fact that mouse and human genomes per se contain a limited number of endogenous targets (so called “attP-pseudosites”).

Additional Ser integrases include: R4, φC31, φBT1, Bxb1, SPBc, TP901-1, Wβ, FC1, φK38, RV, A118, BL3, MR11, TG1 and φ370.

In certain embodiments, the integrase is Bxb1 or φC31.

In certain embodiments, the recombinase is a Bxb1 Ser integrase. The Bxb1 integrase catalyze integration of the Bxb1 bacteriophage genome into the GroEL1 gene of M. segmentis, which contains the attB site. The enzyme does not require supercoiled DNA, and can also work with linear products. The enzyme also works well in human, rat, and mouse cells. Bxb1 integrase yields about two-fold more recombinants and displays about two fold less damage to the recombination sites than recombinase φC31 integrase.

### Hin Recombinase

In certain embodiments, the recombinase is a Hin recombinase, a member of a serine recombinase from the bacteria Salmonella. Hin recombinase is highly similar to gamma-delta resolvase.

The recognition sequences of Hin recombinase (i.e., the Hin binding sites) are two 26-bp imperfect inverted repeat sequences, which are bound by Hin as a homodimer. The Hin binding sites can be inserted into a target DNA to flank a sequence to be inverted. Bacterial accessory proteins Fis binds with nanomolar affinity to the Hin binding sequences, and are required for the recombination to proceed. DNA bending protein HU and divalent metal cation magnesium may also be required.

### Transposases

In certain embodiments, the recombinase is a transposase, such as Transposase Tn5, the Sleeping Beauty (SB) transposase, the piggyBac transposase, the mos transposase, and Transposase Tn7.

Transposons are mobile genetic elements that can excise itself from one genomic location and insert itself into a different genomic location, based on utilizing a transposase that recognizes transposase recognition sequences usually at the ends of the transposon. Thus the transposases can be used as the recombinase of the invention to insert a donor sequence flanked by the transposase recognition sequences into a target DNA, or to excise/delete a target DNA flanked by transposase recognition sequences inserted by the method of the invention.

The transposition of a transposon usually needs just three elements: the transposon (donor DNA), the transposase enzyme, and the target DNA for the insertion of the transposon. Tn5 and most other transposases contain a DDE motif, which is the active site that catalyzes the movement of the transposon. Since transposase is incredibly inactive, the DDE region is mutated to activate transposase—the glutamate (E) is substituted by an aspartate (D) and the two aspartates into glutamates (EED).

In certain embodiments, the recombinase is the Sleeping Beauty (SB) transposase, which is a member of the DD[E/D] family of transposaes that drives the Sleeping Beauty transposon system. SB transposase belongs to a large superfamily of polynucleotidyl transferases that includes RNase H, RuvC Holliday resolvase, RAG proteins, and retroviral integrases. In certain embodiments, the recombinase is the engineered SB100X enzyme that directs high levels of transposon integration.

In certain embodiments, the recombinase is a Tn7 transposase. Tn7 is unique in that it transposes at high-frequency into a single specific site in bacterial chromosomes called attTn7. Thus in certain embodiments, when Tn7 is used in the invention described herein, attTn7 is used as its recognition sequence inserted into the target DNA by the method of the invention, and the donor DNA sequence is flanked by Tn7 recognition sequences for insertion into attTn7.

The Tn7 recognition sequences are two segments located at the ends of the Tn7 transposon. The left segment (Tn7-L) is 150 bp long and the right sequence (Tn7-R) is 90 bp long. Both ends of the transposon contain a series of 22 bp binding sites that the Tn7 transposase recognizes and binds to. The Tn7 transposon encoded TnsA and TnsB interact together to form the Tn7 transposase enzyme TnsAB.

In certain embodiments, the recombinase is a PiggyBac (PB) transposase from the PiggyBac transposon, which is a mobile genetic element that efficiently transposes between vectors and chromosomes via a “cut and paste” mechanism. During transposition, the PB transposase recognizes transposon-specific inverted terminal repeat sequences (ITRs) located on both ends of the transposon vector and efficiently moves the contents from the original sites and efficiently integrates them into TTAA chromosomal sites, which can be inserted into the desired target DNA sequence using the methods of the invention. The powerful activity of the PiggyBac transposon system enables genes of interest between the two ITRs in the PB vector to be easily mobilized into target genomes.

In certain embodiments, the recombinase is a transposase from the Tc1/mariner superfamily of interspersed repeats DNA (Class II) transposons, including the Tc1 transposon of Caenorhabditis elegans and the mariner transposon of Drosophila.

These transposons consist of a transposase gene, flanked by two inverted tandem repeats (TIR). Two short tandem site duplications (TSD) are present on both sides of the insert/transposon. Transposition occurs when two transposases recognize and bind to TIR sequences, join together and promote DNA double-strand cleavage. The DNA-transposase complex then inserts its DNA cargo at specific DNA motifs elsewhere in the genome, creating short TSDs upon integration. In the IS630/Tc1/mariner system, the motif used for insertion is a “TA” dinucleotide, duplicated on both ends after insertion.

Thus in certain embodiments, a donor DNA sequence can be flanked by the two TIR sequences, and the target DNA site can be engineered using the subject pegRNA and methods to include the TA insertion motif, so as to insert the DNA cargo flanked by the TIR sequences into the TA site. Alternatively, a target DNA can be deleted by inserting the two TIR sequences at the ends of the deletion using the methods and pegRNA of the invention.

Exemplary transposases in this family include the Tc1 (DD34E) or Tc3 transposases from Caenorhabditis elegans, the Mariner (DD34D) transposase from Drosophila and many other organisms, such as the human Mariner-like transposons Hsmar1 (cecropia) and Hsmar2 (irritans) subfamilies, Mos1 from Drosophila mauritiana, rosa (DD41D) from Ceratitis rosa, Pogo/Fot1 (DDxD, x indicating a variable length), IS630 from Shigella sonnei, Tigger and other human domestications of pogo include TIGD1, TIGD2, TIGD3, TIGD4, TIGD5, TIGD6, TIGD7, JRK, JRKL, POGK, and POGZ.

In any of the above embodiments, a linker sequence may separate two adjacent recognition sequences. The linker sequence may have a length of from about 3 nucleotides to about 100 nucleotides. For example, the linker sequence can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker sequence can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt.

d. Optional Other Sequences

In some embodiments, the reverse transcriptase for use with the polynucleotide of the invention can be provided as a fusion with the Cas effector enzyme. However, in other embodiment, the reverse transcriptase can also be separately provided in trans and be recruited to the polynucleotide of the invention through binding to a recruitment sequence on the polynucleotide of the invention.

Similarly, the recombinase for use with the polynucleotide of the invention can be provided as a fusion with the Cas effector enzyme. However, in other embodiment, the recombinase can also be provided in trans and be recruited to the polynucleotide of the invention through binding to a recruitment sequence on the polynucleotide of the invention.

Therefore, in certain embodiments, the polynucleotide of the invention may further comprise one or more recruitment sequences for recruiting the reverse transcriptase and/or recombinase provided in trans. The recruitment sequence can be any nucleotide sequence motifs capable of being bound by a protein domain fused to the reverse transcriptase, a recombinase, an integrase or a transposase. For example, the protein domain/recruitment sequence pair can be any one of: MS2 bacteriophage coat protein/a stem-loop structure from the MS2 phage genome (MS2 binding site or MBS); PP7 bacteriophage coat protein (PCP)/PP7 binding site (PBS); or lambdaN Peptide/its specific 19 nt binding site (boxB). Additional protein domain/recruitment sequence pair can include any of the PUF domain/PUF recognition sequences, such as those disclosed in WO2016/148994 (incorporated herein by reference).

A stability control sequence (e.g., transcriptional terminator segment) influences the stability of an RNA (e.g., a subject polynucleotide). One example of a suitable stability control sequence is a transcriptional terminator segment (i.e., a transcription termination sequence). A transcriptional terminator segment of a subject polynucleotide can have a total length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the transcriptional terminator segment can have a length of from about 15 nucleotides (nt) to about 80 nt, from about 15 nt to about 50 nt, from about 15 nt to about 40 nt, from about 15 nt to about 30 nt or from about 15 nt to about 25 nt.

In some cases, a transcription termination sequence is present on the polynucleotide of the invention when it is transcribed from a vector. In certain embodiments, the transcription terminator sequence is an alternative terminator sequence that yields minimal poly A tail. In some embodiments, the transcription termination sequence is one that is functional in a eukaryotic cell. In some other embodiments, the transcription termination sequence is one that is functional in a prokaryotic cell.

Non-limiting examples of additional optional nucleotide sequences that can be included in the polynucleotide of the invention include a stability control sequence (e.g., transcriptional termination segment, or in any segment of the DNA-targeting RNA to provide for increased stability), including sequences set forth in SEQ ID NO: 683-696 of WO 2013/176772 (incorporated herein by reference), see, for example, SEQ ID NO: 795 of WO 2013/176772, a Rho-independent transcription termination site.

Further optional nucleotide sequences that can be included in the polynucleotide of the invention include a sequence that inhibits/deters exonuclease degradation of the polynucleotide. Such sequence may include a Flaviviral Xrn1-resistant structure (which blocks Xrn1's 5′→3′ exonuclease activity), the Kaposi's sarcoma-associated herpesvirus (KSHV) polyadenylated expression and nuclear retention element (PAN ENE) or a similar cellular RNA element found on the 3′ end of the processed metastasis-associated lung adenocarcinoma transcript 1 (MALAT1) RNA (both of which resist 3′-5′ RNA decay by sequestering the 3′ end of the RNA from the unwinding and endonuclease activities of the exosome), and the tRNA-like structure (TLS) found at the extreme 3′ end of the turnip yellow mosaic virus (TYMV), which ability to enhance RNA stability depends on 3′ aminoacylation.

The stability control sequence may be situated after the Cas9-binding sequence, for example, between the Cas9-binding sequence and the recombinase/integrase recognition sequence, or after the primer binding sequence.

In some embodiments, the polynucleotide of the invention or parts thereof (e.g., the DNA-targeting sequence, the binding sequence, the recombinase/integrase recognition sequence, and/or the primer binding sequence, or a polynucleotide encoding the Cas effector enzyme (e.g., wt or nickase) or fusions thereof, or a polynucleotide encoding one of the recombinase/integrase, may comprise a modification or sequence that provides for an additional desirable feature, e.g., modified or regulated stability; subcellular targeting (such as a nuclear localization signal or NLS); tracking (a sequence tag, e.g., 6His tag, FLAG tag, etc.; or a fluorescent label; a binding site for a protein or protein complex; etc.).

Non-limiting examples include: a 5′ cap (e.g., a 7-methylguanylate cap (m⁷G)); a 3′ polyadenylated tail (i.e., a 3′ poly(A) tail); a riboswitch sequence or an aptamer sequence (e.g., to allow for regulated stability and/or regulated accessibility by proteins and protein complexes); a terminator sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates detection including fluorescent detection, a sequence that allows for fluorescent detection, etc.); a modification or sequence that provides a binding site for proteins; a modification or sequence that provides for increased, decreased, and/or controllable stability; and combinations thereof.

### 3. CRISPR-Cas System Effector Enzyme

The polynucleotides, vectors, kits, systems, and methods of the invention can be used with any CRISPR/Cas effector enzyme that is an RNA-guided DNA endonuclease, including a wild-type effector enzyme, or a mutant effector enzyme having the ability to create a single stranded nick on only one strand of the double-stranded target DNA sequence (i.e., a nickase). In some embodiments, the nickase cleaves the non-target strand (NTS) of the effector enzyme, wherein the NTS is the strand that does not anneal to the guide RNA of the effector enzyme, and is the strand that is complementary to the target strand (TS) which anneals to the guide RNA.

In certain embodiments, the effector enzyme is a Type II Class II CRISPR-Cas system effector enzyme. For example, in one embodiment, the effector enzyme is a Cas9, such as SpCas9 from Streptococcus pyogenes, SaCas9 from Staphylococcus aureus, StCas9 from Streptococcus thermophilus, NmCas9 from Neisseria meningitidis, FnCas9 from Francisella novicida, CjCas9 from Campylobacter jejuni, ScCas9 from Streptococcus canis, or a variant thereof (such as eSpCas9, SpCas9-HF1, and xCas9).

In certain embodiments, the Type II Class II CRISPR-Cas system effector enzyme lacks its HNH nuclease activity. In this embodiment, the effector enzyme, such as Cas9, can use its RuvC endonuclease activity to create a nick on the non-target sequence (NTS) which is complementary to the target sequence (TS) recognized by the guide RNA/crRNA. The cleavage by the nickase creates a 5′ end and a 3′ end on the NTS, wherein the free 3′ end can anneal with the primer binding sequence on the polynucleotide of the invention (e.g., a pegRNA).

In certain embodiments, the Type II Class II CRISPR-Cas system effector enzyme is Cas9. The Cas9 protein (e.g., wt, or nickase such as the nickase with RuvC nuclease activity) of the invention comprises: i) an RNA-binding portion that interacts with the binding sequence (in this case, Cas9-binding sequence) of the subject polynucleotide, and ii) an activity portion that exhibits wt, reduced endonuclease (e.g., endodeoxyribonuclease) activity, or lacks one of the two endonuclease activity, depending on the identity of the Cas9 protein.

The Cas9-binding sequence of the polynucleotide and the Cas9 protein (e.g., wt or nickase) can form a complex that binds to a specific target polynucleotide sequence, based on the sequence complementarity between the DNA-targeting sequence and the target polynucleotide sequence. The DNA-targeting sequence of the subject polynucleotide provides target specificity to the complex via its sequence complementarity to the target polynucleotide sequence of a target DNA.

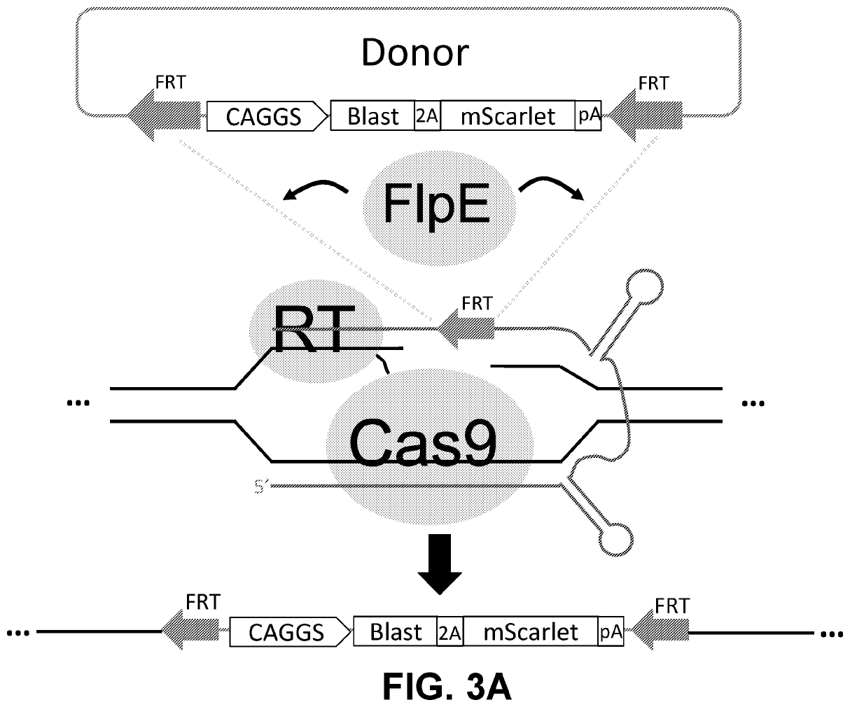

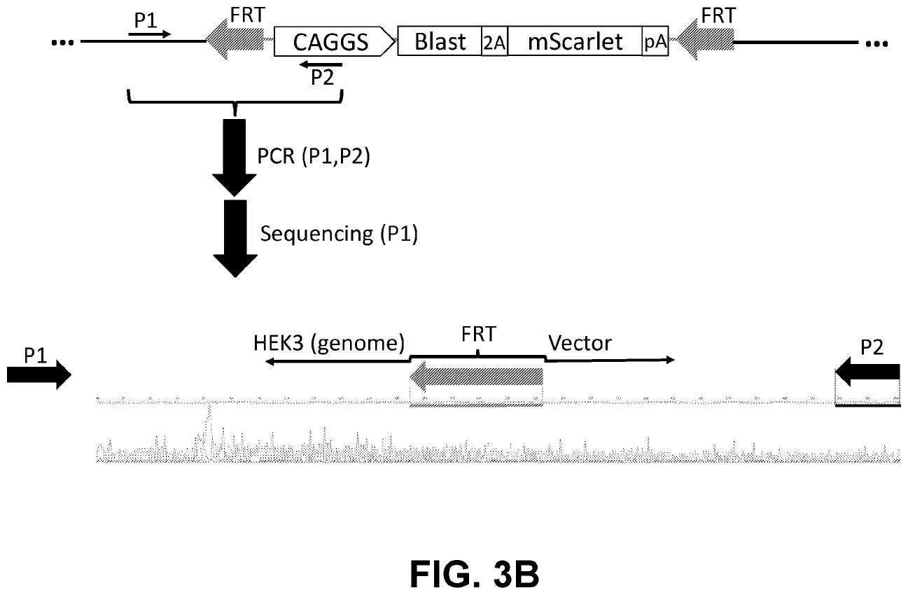

In certain embodiments, the modified Cas9 protein has reduced or lacks endonuclease (e.g., endodeoxyribonuclease) activity. For example, a modified Cas9 suitable for use in a method of the present invention may be a Cas9 nickase, or exhibits less than about 20%, less than about 15%, less than about 10%, less than about 5%, less than about 1%, or less than about 0.1%, of the endonuclease (e.g., endodeoxyribonuclease) activity of a wild-type Cas9 polypeptide, e.g., a wild-type Cas9 polypeptide comprising an amino acid sequence as depicted in FIG. 3 and SEQ ID NO: 8 of WO 2013/176772 (incorporated herein by reference). In some embodiments, a Cas9 variant that can be used in the instant invention is defective in (but may or may not completely lack of) its HNH nuclease activity (e.g., when a Cas9 protein has a E762, H840, N854, N863, H982, H983, A984, D986, and/or a A987 mutation, e.g., E762A, H840A, N854A, N863A, H982A, H983A, A984A, and/or D986A), the polypeptide can still bind to target DNA in a site-specific manner, because it is still guided to a target polynucleotide sequence by a DNA-targeting sequence of the subject polynucleotide, as long as it retains the ability to interact with the Cas9-binding sequence of the subject polynucleotide.

In some cases, a suitable Cas9 protein (e.g., wt or nickase) comprises an amino acid sequence having at least about 75%, at least about 80%, at least about 85%, at least about 90%, at least about 95%, at least about 99% or 100% amino acid sequence identity to amino acids 7-166 or 731-1003 of the Cas9/Csn1 amino acid sequence (of Streptococcus pyogenes), as depicted in FIG. 3 and SEQ ID NO: 8 of WO 2013/176772 (incorporated by reference), or to the corresponding portions in any one of the amino acid sequences SEQ ID NOs: 1-256 and 795-1346 of WO 2013/176772 (incorporated by reference), preferably to the corresponding portions in any one of the amino acid sequences of the orthogonal Cas9 sequences from S. pyogenes, N. meningitidis, S. thermophilus and T. denticola (see, Esvelt et al., Nature Methods, 10(11): 1116-1121, 2013, incorporated by reference).

In some cases, the Cas9 nickase can cleave the non-complementary strand (NTS) of the target DNA but has reduced or no ability to cleave the complementary strand (TS) of the target DNA. For example, the Cas9 nickase can have a mutation (amino acid substitution) that reduces the function of the HNH domain (RuvC/HNH/RuvC domain motifs). As a non-limiting example, in some cases, the Cas9 nickase is a H840A (histidine to alanine at amino acid position 840 of SEQ ID NO: 8 of WO 2013/176772, incorporated by reference) or the corresponding mutation of any of the amino acid sequences set forth in SEQ ID NOs: 1-256 and 795-1346 of WO 2013/176772 (all such sequences incorporated by reference).

Other residues can be mutated to achieve the same effect (i.e. inactivate one or the other nuclease portions). As non-limiting examples, residues E762, H840, N854, N863, H982, H983, A984, D986, and/or A987 (or the corresponding mutations of any of the proteins set forth as SEQ ID NOs: 1-256 and 795-1346) can be altered (i.e., substituted) (see FIGS. 3, 5, 11A, and Table 1 of WO 2013/176772 (all incorporated by reference) for more information regarding the conservation of Cas9 amino acid residues). Also, mutations other than alanine substitutions (such as conservative substitutions that may only reduce rather than eliminate nuclease activity) may be suitable.

Specific Cas effector enzymes has specific requirements for PAM (protospacer adjacent motif) sequences adjacent to the target strand sequence for cleavage. Thus in certain embodiments, the PAM sequence of the complementary strand (TS) matches the specific Cas9 protein or homologs or orthologs to be used.

In certain embodiments, the target polynucleotide sequence is immediately 3′ to a PAM sequence of the complementary strand (TS). For example, in certain embodiments, the PAM sequence of the complementary strand is 5′-CCN-3′, wherein N is any DNA nucleotide.

As is known in the art, for Cas9 to successfully bind to DNA, the target sequence in the genomic DNA must be complementary to the guide RNA sequence and must be immediately followed by the correct protospacer adjacent motif or PAM sequence. The PAM sequence is present in the DNA target sequence but not in the guide RNA sequence. Any DNA sequence with the correct target sequence followed by the PAM sequence will be bound by Cas9.

The PAM sequence varies by the species of the bacteria from which the Cas9 was derived. The most widely used Type II CRISPR system is derived from S. pyogenes and the PAM sequence is 5′-NGG-3′ located on the immediate 3′ end of the guide RNA recognition sequence (or 5′-CCN-3′ on the complementary strand). The PAM sequences of other Type II CRISPR systems from different bacterial species are listed in the Table below.

| | |
|---|---|
| Streptococcus pyogenes (SP) | NGG |
| Neisseria meningitidis (NM) | NNNNGATT |
| Streptococcus thermophilus (ST) | NNAGAA |
| Treponema denticola (TD) | NAAAAC |

In certain embodiments, the DNA-targeting sequence base-pairs with the target polynucleotide sequence when the Cas9 protein (e.g., wt or nickase) is complexed with the polynucleotide.

It should be noted that the DNA-targeting sequence may or may not be 100% complementary to the target polynucleotide sequence. In certain embodiments, the DNA-targeting sequence is complementary to the target polynucleotide sequence over about 8-25 nucleotides (nts), about 12-22 nucleotides, about 14-20 nts, about 16-20 nts, about 18-20 nts, or about 8, 9, 10, 11, 12, 13, 14, 15, 16, 17, 18, 19, 20, 21, 22, 23, 24, or 25 nts. In certain embodiments, the complementary region comprises a continuous stretch of about 12-22 nts, preferably at the 3′ end of the DNA-targeting sequence. In certain embodiments, the 5′ end of the DNA-targeting sequence has up to 8 nucleotide mismatches with the target polynucleotide sequence. In certain embodiments, the DNA-binding sequence is about 50, 55, 60, 65, 70, 75, 80, 85, 90, 95, or 100% complementary to the target polynucleotide sequence.

In a related embodiment, there is no more than 15-nucleotide match at the 3′ end of the DNA-targeting sequence compared to the complementary target polynucleotide sequence, and the Cas9 protein in the complex is a wt Cas9 protein which, under the circumstance, binds but does not cut a target DNA.

In certain embodiments, the DNA-binding sequence has a free 5′ end nucleotide G.

In certain embodiments, the polynucleotide further comprises a linker sequence linking the DNA-targeting sequence to the Cas9-binding sequence.

In certain embodiments, the Cas9-binding sequence forms a hairpin structure. In certain embodiments, the Cas9-binding sequence is about 30-100 nt, about 35-50 nt, about 37-47 nt, or about 42 nt in length.

An exemplary Cas9-binding sequence is GTTTTAGAGCTAGAAATAGCAAGTTAA AATAAGGCTA. Another exemplary Cas9-binding sequence is GTTTAAGAGCTATGCTG GAAACAGCATAGCAAGTTTAAATAAGGCTA.

The modified Cas9 protein (e.g., a nickase) may have reduced nuclease activity, or lacks nuclease activity at one of the endonuclease catalytic sites (e.g., HNH). For example, the point mutations may be H840A, in the S. pyogenes Cas9, or in the corresponding residues in species other than S. pyogenes.

In certain embodiments, the Type II Class II CRISPR-Cas system effector enzyme binds to a binding sequence of the polynucleotide of the invention, wherein the binding sequence is 3′ to the DNA-targeting sequence or spacer sequence that is complementary to the target strand. In one embodiment, the binding sequence is 5′ to the integrase or recombinase recognition sequence or complement thereof, which is 5′ to the primer binding sequence. In another embodiment, the DNA-targeting sequence (spacer sequence) is 3′ to the primer binding sequence, which is 3′ to the integrase or recombinase recognition sequence or complement thereof.

In certain embodiments, the Type II Class II CRISPR-Cas system effector enzyme binds to a binding sequence of the polynucleotide of the invention, wherein the binding sequence is 5′ to the DNA-targeting sequence or spacer sequence that is complementary to the target strand. In one embodiment, the DNA-targeting (spacer) sequence is 5′ to the integrase or recombinase recognition sequence or complement thereof, which is 5′ to the primer binding sequence. In another embodiment, the binding sequence is 3′ to the primer binding sequence, which is 3′ to the integrase or recombinase recognition sequence or complement thereof.

In certain embodiments, the effector enzyme is a Type V Class II CRISPR-Cas system effector enzyme. Such effector enzymes may lack HNH endonuclease activity, but may possess other endonuclease activity (such as the Nuc endonuclease activity in Cpf1). For example, in one embodiment, the effector enzyme is Cpf1, C2c1, or C2c3.

In one embodiment, the effector enzyme is Cpf1, such as one from Francisella novicida U112, Prevotella disiens, Acidaminococcus sp. BV3L6, Lachnospiraceae bacterium ND2006, Lachnospiraceae bacterium MA2020, Candidatus Methanoplasma termitum, Moraxella bovoculi 237, or Porphyromonas crevioricanis.

In one embodiment, the effector enzyme is Cpf1, such as Acidaminococcus sp. BV3L6 Cpf1 (AsCpf1), Francisella novicida U112 Cpf1 (FnCpf1), Moraxella bovoculi Cpf1 (MbCpf1), Porphyromonas crevioricanis Cpf1 (PcCpf1), or Lachnospiraceae bacterium ND2006 Cpf1 (LbCpf1), or a variant thereof. In one embodiment, the variant is an AsCpf1 variant, have either the mutations S542R/K607R or S542R/K548V/N552R, which recognize TYCV and TATV PAMs, respectively. These variants have enhanced activities in vitro and in human cells.

In certain embodiments, the Type V Class II CRISPR-Cas system effector enzyme is a nickase, e.g., one with only RuvC or RuvC-like endonuclease activity. The cleavage by the nickase creates a 5′ end and a 3′ end on the NTS, wherein the free 3′ end can anneal with the primer binding sequence on the polynucleotide of the invention (e.g., a pegRNA).

In certain embodiments, the Type V Class II CRISPR-Cas system effector enzyme binds to a binding sequence of the polynucleotide of the invention, wherein the binding sequence is 5′ to the DNA-targeting sequence or spacer sequence that is complementary to the target strand. In one embodiment, the DNA-targeting (spacer) sequence is 5′ to the integrase or recombinase recognition sequence or complement thereof, which is 5′ to the primer binding sequence. In another embodiment, the binding sequence is 3′ to the primer binding sequence, which is 3′ to the integrase or recombinase recognition sequence or complement thereof.

In certain embodiments, the Type V Class II CRISPR-Cas system effector enzyme binds to a binding sequence of the polynucleotide of the invention, wherein the binding sequence is 3′ to the DNA-targeting sequence or spacer sequence that is complementary to the target strand. In one embodiment, the binding sequence is 5′ to the integrase or recombinase recognition sequence or complement thereof, which is 5′ to the primer binding sequence. In another embodiment, the DNA-targeting sequence (spacer sequence) is 3′ to the primer binding sequence, which is 3′ to the integrase or recombinase recognition sequence or complement thereof.

In certain embodiments, the effector enzyme (e.g., wt or nickase) is optionally fused to one or more functional domains.

In one embodiment, the functional domain comprises a reverse transcriptase domain that can direct the synthesis (reverse transcription) of cDNA, using the primer sequence generated by the RuvC cleavage and anneled to the primer binding sequence as a primer, and using the integrase or recombinase recognition sequence or complement thereof as the template sequence for cDNA synthesis.

In one embodiment, the functional domain comprises a recombinase or integrase that can facilitate the integration of a donor DNA into the modified target DNA sequence having incorporated one or more integrase or recombinase recognition sequence or complement thereof.

In one embodiment, the functional domain comprises a recombinase or integrase, as well as a reverse transcriptase.

In one embodiment, the functional domain is fused N-terminal to the effector enzyme.

In one embodiment, the functional domain is fused C-terminal to the effector enzyme.

In one embodiment, one functional domain is fused N-terminal to the effector enzyme, and another functional domain is fused C-terminal to the effector enzyme.

The effector enzyme and the functional domain(s) may be linked by a linker sequence of appropriate length to provide the beneficial orientation and flexibility between the effector enzyme and the functional domain(s).

### 4. Vectors

Another aspect of the invention provides a vector encoding any one of the subject polynucleotide. In certain embodiments, transcription of the polynucleotide is under the control of a constitutive promoter, or an inducible promoter. In certain embodiments, the vector is active in a cell from a mammal (a human; a non-human primate; a non-human mammal; a rodent such as a mouse, a rat, a hamster, a Guinea pig; a livestock mammal such as a pig, a sheep, a goat, a horse, a camel, cattle; or a pet mammal such as a cat or a dog); a bird, a fish, an insect, a worm, a yeast, or a bacterium.

In certain embodiments, the vector is a plasmid, a viral vector (such as adenoviral, retroviral, or lentiviral vector, or AAV vector), or a transposon (such as piggyBac transposon). The vector can be transiently transfected into a host cell, or be integrated into a host genome by infection or transposition.

A related aspect of the invention provides a plurality or a library of any one of the vectors of the invention, wherein two of the vectors differ in the encoded polynucleotides in their respective DNA-targeting sequences, CRISPR/Cas effector enzyme-binding sequences, and/or recombinase or integrase recognition sequences and/or the copy number, orientation thereof. Optionally, the vector may also differ in the sequence, length, degree of sequence complementarity between the primer-binding sequence and the primer sequence on the TS.

### 5. Complexes

Another aspect of the invention provides a complex comprising any one of the polynucleotide of the invention, and a fusion protein comprising: (i) a Type II or Type V Class II CRISPR-Cas system effector enzyme lacking HNH nuclease activity; and, (ii) a reverse transcriptase; wherein the polynucleotide is complexed with the effector enzyme lacking HNH nuclease activity through the binding sequence.

A related/alternative aspect of the invention provides a complex comprising any one of the polynucleotide of the invention, and a Type II or Type V Class II CRISPR-Cas system effector enzyme lacking HNH nuclease activity; wherein the polynucleotide is complexed with the effector enzyme lacking HNH nuclease activity through the binding sequence.

Yet another alternative aspect of the invention provides a complex comprising any one of the polynucleotide of the invention, and a fusion protein comprising: (i) a Type II or Type V Class II CRISPR-Cas system effector enzyme lacking HNH nuclease activity; (ii) a reverse transcriptase; and (iii) an integrase or recombinase compatible with the integrase/recombinase recognition sequence on the polynucleotide of the invention, wherein the polynucleotide is complexed with the effector enzyme lacking HNH nuclease activity through the binding sequence.

Another aspect of the invention provides a complex comprising: (a) any one of the polynucleotide of the invention, and, (b) a CRISPR-Cas system effector enzyme (such as a Type II or Type V Class II CRISPR-Cas system effector enzyme) lacking HNH or equivalent nuclease activity; wherein the polynucleotide of the invention further comprises a recruitment sequence for a reverse transcriptase, a recombinase, an integrase or a transposase; wherein the polynucleotide (i) is complexed with the effector enzyme lacking HNH or equivalent nuclease activity through the binding sequence, and (ii) is complexed with the reverse transcriptase, recombinase, integrase or transposase through the recruitment sequence.

Another aspect of the invention provides a complex comprising: (a) any one of the polynucleotide of the invention, and, (b) a CRISPR-Cas system effector enzyme (such as a Type II or Type V Class II CRISPR-Cas system effector enzyme) lacking HNH or equivalent nuclease activity; wherein the complex further comprises one or more of (1) a reverse transcriptase; and (2) a recombinase, an integrase or a transposase; wherein (1) or (2) or both is/are either fused to the Cas effector enzyme as a fusion protein, or is/are recruited to the polynucleotide of the invention through protein domain/recruitment sequence pair (wherein the protein domain is fused to (1) and/or (2), and wherein the recruitment sequence is in the polynucleotide of the invention). In this configuration, the polynucleotide of the invention (i) is complexed with the effector enzyme lacking HNH or equivalent nuclease activity through the binding sequence, and (ii) is complexed with the reverse transcriptase, recombinase, integrase or transposase through the recruitment sequence, if the reverse transcriptase, recombinase, integrase or transposase is not already fused with the Cas effector enzyme.

In any of the above variations, the Cas effector enzyme may be fused with the reverse transcriptase and/or the integrase/recombinase in any orientation, if the Cas effector enzyme is fused to the reverse transcriptase and/or the integrase/recombinase at all (e.g., the Cas effector enzyme may not be fused to the reverse transcriptase and/or the integrase/recombinase which can be provided in trans). For example, the Cas effector enzyme may be fused to the reverse transcriptase and/or the integrase/recombinase at the N—, C—, or both termini of the effector enzyme. In embodiments in which both reverse transcriptase and the integrase/recombinase are fused with the Cas effector enzyme, the reverse transcriptase and the integrase/recombinase may be both fused to the N- or C-terminus of the Cas effector enzyme, in any order. Alternatively, the reverse transcriptase and the integrase/recombinase may be fused to the N- and C-terminus (or C- and N-terminus) of the Cas effector enzyme, respectively.

In certain embodiments, more than one integrase/recombinase may be in the fusion. In this embodiment, the more than one integrase/recombinase may be identical or different.

In any of the above variations, the complex may further comprise: (c) a double-stranded target DNA sequence comprising a target strand and a complementary non-target strand; wherein the DNA-targeting sequence of the polynucleotide binds to the target strand; and, wherein the effector enzyme is capable of cleaving the non-target strand through the RuvC (or RuvC like) nuclease activity to release the 3′-end of the primer sequence, for priming reverse transcription by the reverse transcriptase, upon binding of the primer sequence to the complementary sequence to integrate the integrase or recombinase recognition sequence into the reverse transcription transcript.

In certain embodiments, the complex comprises any one of the polynucleotide of the invention, and the Cas9 or Cpf1 protein (e.g., wt or nickase). In certain embodiments, the complex does not comprise the wt Cas9 protein or the wt Cpf1 protein. In certain embodiments, the complex comprises the Cas9 or Cpf1 nickase (e.g., one has RuvC activity).

In certain embodiments, the complex may further comprise one or more integrase/recombinase and/or reverse transcriptase, when the integrase/recombinase and/or reverse transcriptase is not fused to the Cas effector enzyme. The integrase/recombinase and/or reverse transcriptase may be recruited to the complex by recruitment sequences on the subject polynucleotide (e.g., RNA), and/or on the anneled subject polynucleotide and the target strand (TS), and/or the annealed subject polynucleotide and the primer sequence.

In certain embodiments, the Cas effector enzyme (e.g., wt or nickase), or the fusion protein thereof, further comprises a nuclear localization signal (NLS).

In certain embodiments, the complex is bound to the target polynucleotide sequence through the DNA-targeting sequence of the polynucleotide.

Any and all features and limitation concerning the Cas effector enzyme as described above are expressly incorporate herein by reference, but not explicitly recited to avoid redundancy. For example, in certain embodiments, the effector enzyme is a Cas9 nickase that lacks HNH endonuclease activity due to a point mutation at the endonuclease catalytic site of the HNH endonuclease. The Cas9 nickase may comprises a point mutation H840A or equivalent thereof.

Yet another aspect of the invention provides a method of assembling the complex of the invention at the target polynucleotide sequence, the method comprising contacting or bringing to the vicinity of the target polynucleotide sequence: (1) any one of the subject polynucleotide, or any one of the subject vector, or the plurality of vectors; (2) the Type II or Type V Class II CRISPR-Cas system effector enzyme, such as Cas9 or Cpf1 (e.g., wt or nickase), including fusions thereof with the recombinase/integrase/reverse transcriptase (if any), or any one of the subject second vector encoding the effector enzyme (e.g., wt or nickase) or fusions thereof; and, (3) a donor sequence flanked by compatible recognition sequences for the recombinase/integrase, optionally on a third vector.

In certain embodiments, the complex is assembled inside a cell, the target polynucleotide sequence is a part of the genomic DNA of the cell, and wherein the subject vector, second vector, and third vector are introduced into the cell.

In certain embodiments, the target polynucleotide sequence is at, within, or adjacent to a target gene of interest (GOI). For example, the GOI may be a defective gene, a disease gene, or a wild-type or mutant gene desired to be inactivated.

In certain embodiments, the target DNA sequence is within an intron, such as the first intron of the GOI. For example, the GOI may be a disease/mutant gene, which may have a single mutation, or multiple mutations located in different introns and/or exons.

In certain embodiments, the method and system of the invention is used to target an intron immediate upstream of the exon harboring the first mutation.

In certain embodiments, the GOI is a large gene (e.g., more than 5 kb, 10 kb, 20 kb, 50 k, 100 kb, 200 kb, 500 kb, 750 kb, 1 mb, 1.2 mb, 1.5 mb, 2 mb, or about 2.5 mb).

In certain embodiments, the GOI has about or more than 10 exons, 20 exons, 30 exons, 40 exons, 50 exons, 60 exons, 70 exons, or about or more than 80 exons.

In certain embodiments, a portion of the GOI is replaced, such as the portion of the GOI having the one or more mutations, by inserting the wide-type copy of the exons-CDS and using splice acceptor site to restore correct host gene splicing to create a functional or wild-type GOI.

In certain embodiments, the GOI has multiple mutations, including mutations not at the first exon, and a portion of the GOI is inserted at an intron immediate upstream of the exon harboring the first mutation in order to restore wt GOI function.

In certain embodiments, the target DNA sequence comprises or is adjacent to a transcription regulatory element. In certain embodiments, the transcription regulatory element comprises one or more of: core promoter, proximal promoter element, enhancer, silencer, insulator, and locus control region.

In certain embodiments, the target DNA sequence comprises or is adjacent to a telomere sequence, a centromere, or a repetitive genomic sequence.

In certain embodiments, the target DNA sequence comprises or is adjacent to a genomic marker sequence (or a genomic locus of interest).

In certain embodiments, transcription of the target DNA sequence, for example, may affect cell fate determination, cell differentiation, metabolic flux, or a biologically or biochemically determinable outcome.

### 6. Host Cells

Another aspect of the invention provides a host cell comprising any one of the subject vector, or the plurality of vectors, that encoding one or more polynucleotide(s) of the invention, e.g., pegRNA.

In certain embodiments, the host cell further comprises a second vector encoding a CRISPR/Cas effector enzyme, such as a Type II or V class 2 CRISPR/Cas effector enzyme.

In certain embodiments, the effector enzyme is Cas9 (e.g., wt or nickase). In certain embodiments, the second vector further encodes an effector domain fused to the Cas9 protein (e.g., wt or nickase). The expression of the Cas9 protein (e.g., wt or nickase) can be under the control of a constitutive promoter or an inducible promoter.

Any and all features and limitation concerning the Cas effector enzyme and fusions thereof with recombinase, integrase, and/or reverse transcriptase, as described above, are expressly incorporate herein by reference, but not explicitly recited to avoid redundancy. For example, in certain embodiments, the effector enzyme is a Cas9 nickase that lacks HNH endonuclease activity due to a point mutation at the endonuclease catalytic site of the HNH endonuclease. The Cas9 nickase may comprises a point mutation H840A or equivalent thereof.

In certain embodiments, the host cell may further comprise a third vector encoding the one or more functional domains, to the extent that such functional domains are not fused to the Cas effector enzyme. For example, the third vector may encode one or more recombinase/integrase/reverse transcriptase.

In certain embodiments, the host cell may further comprise a fourth vector comprising a donor polynucleotide or donor sequence to be inserted into the target sequence. The donor sequence may have flanking recombinase/integrase recognition sequences that can be used by recombinase/integrase for insertion into the target sequence modified by the pegRNA-mediated primer editing.

In certain embodiments, the donor sequence, flanked by compatible integrase or recombinase recognition sequences that can direct the integration of the donor sequence into target DNA sequence comprising the integrase or recombinase recognition sequences, is at least about 100 bp, 150 bp, 200 bp, 500 bp, 1 kb, 1.5 kb, 2, kb, 3 kb, 4 kb, 5 kb, 6 kb, 8 kb, or 10 kb.

The donor sequence can encode anything, with or without a biological function (including a scrambled control sequence). In certain embodiments, the donor sequence may encode a protein with any of many functions or biological effects. Merely to illustrate, the encoded protein can be a wild-type or functional protein that can be inserted into a genomic locus to replace the lost function of a mutant gene, which may be a structural protein, an enzyme, a transcription repressor, a transcription activator, a fluorescent protein, or a chromatin remodeling protein (HDAC/HAT), etc.

In certain embodiments, sequences that can be encoded by different vectors may be on the same vector. For example, in certain embodiments, the second vector may be the same as the first vector, and/or the third vector may be the same as the first vector or the second vector.

The expression of the one or more Cas effector enzymes, recombinase, integrase, or reverse transcriptase, or fusions thereof; and the polynucleotide of the invention (e.g., pegRNA having recombinase/integrase recognition sequences), can be independently under the control of a constitutive promoter or an inducible promoter; a ubiqutous promoter or a tissue specific promoter.

In certain embodiments, the host cell is in a live animal. In certain embodiments, the host cell is a cultured cell.

In certain embodiments, the host cell is a mitotic cell. In certain embodiments, the host cell is a post-mitotic cells.

Suitable host cells include, but are not limited to, a bacterial cell; an archaeal cell; a single-celled eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell; an animal cell; a cell from an invertebrate animal (e.g., an insect, a cnidarian, an echinoderm, a nematode, etc.); a eukaryotic parasite (e.g., a malarial parasite, e.g., Plasmodium falciparum; a helminth; etc.); a cell from a vertebrate animal (e.g., fish, amphibian, reptile, bird, mammal); a mammalian cell, e.g., a rodent cell, a human cell, a non-human primate cell, etc. Suitable host cells include naturally-occurring cells; genetically modified cells (e.g., cells genetically modified in a laboratory, e.g., by the “hand of man”); and cells manipulated in vitro in any way. In some cases, a host cell is isolated or cultured.

Any type of cell may be of interest (e.g., a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells,” “primary cell lines,” and “primary cultures” are used interchangeably herein to refer to cells and cells cultures that have been derived from a subject and allowed to grow in vitro for a limited number of passages, i.e. splittings, of the culture. For example, primary cultures include cultures that may have been passaged 0 times, 1 time, 2 times, 4 times, 5 times, 10 times, or 15 times, but not enough times go through the crisis stage. Primary cell lines can be are maintained for fewer than 10 passages in vitro. Target cells are in many embodiments unicellular organisms, or are grown in culture.

If the cells are primary cells, such cells may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, e.g., from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% dimethyl sulfoxide (DMSO), 50% serum, 40% buffered medium, or other solutions commonly used in the art to preserve cells at such freezing temperatures, and thawed in a manner as commonly known in the art for thawing frozen cultured cells.

In certain embodiments, the host cell is a normal or healthy cell. In certain embodiments, the host cell is a diseased cell or from a disease tissue.

### 7. Introducing Nucleic Acid into a Host Cell

A subject polynucleotide, a nucleic acid comprising a nucleotide sequence encoding same (e.g., a vector of the invention), or a nucleic acid comprising a nucleotide sequence encoding the subject Cas effector enzyme such as Cas9 or Cpf1 (e.g., wt or nickase) or a fusion thereof with a recombinase/integrase/reverse transcriptase, can be introduced into a host cell by any of a variety of well-known methods.

Methods of introducing a nucleic acid into a host cell are known in the art, and any known method can be used to introduce a nucleic acid (e.g., vector or expression construct) into a stem cell or progenitor cell. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv. Drug Deliv. Rev., pii: S0169-409X(12)00283-9.doi:10.1016/j.addr.2012.09.023), and the like.

Thus the present invention also provides an isolated nucleic acid comprising a nucleotide sequence encoding a subject polynucleotide. In some cases, a subject nucleic acid also comprises a nucleotide sequence encoding a subject Cas effector enzyme such as Cas9 or Cpf1 (e.g., wt or nickase) and/or a fusion thereof.

In some embodiments, a subject method involves introducing into a host cell (or a population of host cells) one or more nucleic acids (e.g., vectors) comprising nucleotide sequences encoding a subject polynucleotide and/or a subject Cas effector enzyme (e.g., wt or nickase) and/or a fusion thereof. In some embodiments a host cell comprising a target DNA is in vitro. In some embodiments a host cell comprising a target DNA is in vivo. Suitable nucleic acids comprising nucleotide sequences encoding a subject polynucleotide and/or a subject Cas effector enzyme (e.g., wt or nickase) and/or a fusion thereof include expression vectors, where the expression vectors may be recombinant expression vector.

In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g. viral vectors based on vaccinia virus; poliovirus; adenovirus (see, e.g., Li et al., Invest Opthalmol. Vis. Sci., 35:2543-2549, 1994; Borras et al., Gene Ther., 6:515-524, 1999; Li and Davidson, Proc. Natl. Acad. Sci. USA, 92:7700-7704, 1995; Sakamoto et al., Hum. Gene Ther., 5:1088-1097, 1999; WO 94/12649, WO 93/03769; WO 93/19191; WO 94/28938; WO 95/11984 and WO 95/00655); adeno-associated virus (see, e.g., Ali et al., Hum. Gene Ther., 9:81-86, 1998, Flannery et al., Proc. Natl. Acad. Sci. USA, 94:6916-6921, 1997; Bennett et al., Invest Opthalmol Vis Sci 38:2857-2863, 1997; Jomary et al., Gene Ther., 4:683-690, 1997, Rolling et al., Hum. Gene Ther., 10:641-648, 1999; Ali et al., Hum. Mol. Genet., 5:591-594, 1996; Srivastava in WO 93/09239, Samulski et al., J. Vir., 63:3822-3828, 1989; Mendelson et al., Virol., 166: 154-165, 1988; and Flotte et al., Proc. Natl. Acad. Sci. USA, 90: 10613-10617, 1993); SV40; herpes simplex virus; human immunodeficiency virus (see, e.g., Miyoshi et al., Proc. Natl. Acad. Sci. USA, 94: 10319-23, 1997; Takahashi et al., J. Virol., 73:7812-7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, HIV virus, myeloproliferative sarcoma virus, and mammary tumor virus); and the like.

Numerous suitable expression vectors are known to those skilled in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al., Methods in Enzymology, 153:516-544, 1987).

In some embodiments, a nucleotide sequence encoding a subject polynucleotide and/or a subject Cas effector enzyme such as Cas9 of Cpf1 (e.g., wt or nickase) and/or a fusion thereof is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, a nucleotide sequence encoding a subject polynucleotide and/or a subject Cas effector enzyme (e.g., wt or nickase) and/or a fusion thereof is operably linked to multiple control elements that allow expression of the nucleotide sequence encoding the subject polynucleotide and/or a subject Cas effector enzyme such as Cas9 or Cpf1 (e.g., wt or nickase) and/or a fusion thereof in both prokaryotic and eukaryotic cells.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotech., 20:497-500, 2002), an enhanced U6 promoter (e.g., Xia et al., Nucleic Acids Res., 31(17):e100, 2003), a human HI promoter (HI), and the like.

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), Steroid-regulated promoter, Metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a subject Cas effector enzyme such as Cas9 or Cpf1 (e.g., wt or nickase) or a fusion thereof in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al., Cell, 51:7-19, 1987; and Llewellyn et al., Nat. Med., 16(10): 1161-1166, 2010); a serotonin receptor promoter (see, e.g., GenBank S62283); a tyrosine hydroxylase promoter (TH) (see, e.g., Oh et al., Gene Ther., 16:437, 2009; Sasaoka et al., Mol. Brain Res., 16:274, 1992; Boundy et al., Neurosci., 18:9989, 1998; and Kaneda et al., Neuron, 6:583-594, 1991); a GnRH promoter (see, e.g., Radovick et al., Proc. Natd. Acad. Sci. USA, 88:3402-3406, 1991); an L7 promoter (see, e.g., Oberdick et al., Science, 248:223-226, 1990); a DNMT promoter (see, e.g., Bartge et al., Proc. Natd. Acad. Sci. USA, 85:3648-3652, 1988); an enkephalin promoter (see, e.g., Comb et al., EMBO J., 17:3793-3805, 1988); a myelin basic protein (MBP) promoter; a Ca²⁺-calmodulin-dependent protein kinase II-alpha (CamKIIa) promoter (see, e.g., Mayford et al., Proc. Natd. Acad. Sci. USA, 93: 13250, 1996; and Casanova et al., Genesis, 31:37, 2001); a CMV enhancer/platelet-derived growth factor-β promoter (see, e.g., Liu et al., Gene Therapy, 11:52-60, 2004); and the like.

Adipocyte-specific spatially restricted promoters include, but are not limited to aP2 gene promoter/enhancer, e.g., a region from −5.4 kb to +21 bp of a human aP2 gene (see, e.g., Tozzo et al., Endocrinol. 138: 1604, 1997; Ross et al., Proc. Natd. Acad. Sci. USA, 87:9590, 1990; and Pavjani et al., Nat. Med., 11:797, 2005); a glucose transporter-4 (GLUT4) promoter (see, e.g., Knight et al., Proc. Natd. Acad. Sci. USA, 100: 14725, 2003); a fatty acid translocase (FAT/CD36) promoter (see, e.g., Kuriki et al., Biol. Pharm. Bull., 25: 1476, 2002; and Sato et al., Biol. Chem. 277: 15703, 2002); a stearoyl-CoA desaturase-1 (SCD1) promoter (Tabor et al., Biol. Chem. 274:20603, 1999); a leptin promoter (see, e.g., Mason et al., Endocrinol. 139: 1013, 1998; and Chen et al., Biochem. Biophys. Res. Comm., 262: 187, 1999); an adiponectin promoter (see, e.g., Kita et al., Biochem. Biophys. Res. Comm., 331:484, 2005; and Chakrabarti, Endocrinol. 151:2408, 2010); an adipsin promoter (see, e.g., Piatt et al., Proc. Natl. Acad. Sci. USA, 86:7490, 1989); a resistin promoter (see, e.g., Seo et al., Molec. Endocrinol., 17: 1522, 2003); and the like.

Cardiomyocyte-specific spatially restricted promoters include, but are not limited to control sequences derived from the following genes: myosin light chain-2, a-myosin heavy chain, AE3, cardiac troponin C, cardiac actin, and the like. Franz et al., Cardiovasc. Res., 35:560-566, 1997; Robbins et al., Ann. N.Y. Acad. Sci., 752:492-505, 1995; Linn et al., Circ. Res., 76:584-591, 1995; Parmacek et al., Mol. Cell. Biol., 14:1870-1885, 1994; Hunter et al., Hypertension, 22:608-617, 1993; and Sartorelli et al., Proc. Natl. Acad. Sci., 89:4047-4051, 1992.

Smooth muscle-specific spatially restricted promoters include, but are not limited to an SM22a promoter (see, e.g., Akyurek et al., Mol. Med., 6:983, 2000; and U.S. Pat. No. 7,169,874); a smoothelin promoter (see, e.g., WO 2001/018048); an a-smooth muscle actin promoter; and the like. For example, a 0.4 kb region of the SM22a promoter, within which lie two CArG elements, has been shown to mediate vascular smooth muscle cell-specific expression (see, e.g., Kim et al., Mol. Cell. Biol., 17:2266-2278, 1997; Li et al., J. Cell Biol., 132:849-859, 1996; and Moessler et al., Development, 122:2415-2425, 1996).

Photoreceptor-specific spatially restricted promoters include, but are not limited to, a rhodopsin promoter; a rhodopsin kinase promoter (Young et al., Ophthalmol. Vis. Sci., 44:4076, 2003); a beta phosphodiesterase gene promoter (Nicoud et al., Gene Med., 9: 1015, 2007); a retinitis pigmentosa gene promoter (Nicoud et al., 2007, supra); an interphotoreceptor retinoid-binding protein (IRBP) gene enhancer (Nicoud et al. (2007) supra); an IRBP gene promoter (Yokoyama et al., Exp. Eye Res., 55:225, 1992); and the like.

In certain embodiments, the polynucleotide of the invention comprising the guide RNA sequence for the cas effector is encoded by a vector of the invention, and is transcribed from the vector under the control of a promoter. In certain embodiments, the promoter is a Pol III promoter, such as a constitutively active U6 promoter. In other embodiment, the promoter is a Pol II promoter, such as any one of the Pol II promoter described herein, including a Pol II promoter subject to spacial/temporal control or regulation. A Pol II promoter-expressed subject polynucleotide can be rapidly modified with a 5′ cap and poly-A tail, and can be undesirably exported from the nucleus to prevent efficient use of the encoded guide RNA-containing polynucleotide of the invention. Thus in certain embodiments, the subject polynucleotide can be further modified to prolong its stay inside the nucleus. For example, in certain embodiment, the subject polynucleotide may be transcribed from an alternative transcriptional terminators that replace the standard poly A-producing ternimating sequence, and include the histone 1h3h terminator, a minimal poly A sequence, the MALAT 1 terminator, or the U1 snoRNA 3′ box (Shechner et al., Nat. Methods 12:664-670, 2015, incorporated by reference). In certain embodiments, the subject polynucleotide may be embedded in a spliced intron, such as an artificial intron inside the mKate fluorphore (Kiani et al., Nat. Methods 11:723-726, 2014, incorporated by reference). In certain embodiments, the subject polynucleotide further comprises a self-cleaving ribozyme-based release system, such as the flanking hammerhead and HDV ribozymes (Nissim et al., Mol. Cell 54:698-710, 2014; and Xu et al., Nucleic Acids Res. doi.org/10.1093/nar/gkw1048, 2016; both incorporated by reference). In certain embodiments, the subject polynucleotide is excised from the Pol II transcript using Csy4 or an orthologous ribonuclease that releases the guide RNA-containing polynucleotide from the flanking Csy4 target sequences (Nissim, supra). In yet other embodiments, the subject polynucleotide is transcribed from a Pol II promoter in a tRNA scaffold (Knapp et al., Nat Commun 10:1490, 2019, incorporated by reference).

### 8. Libraries

The present invention also provides a plurality or library of the subject polynucleotide sequences, or a plurality or library of the vectors encoding the same. The latter may comprise a library of recombinant expression vectors comprising nucleotides encoding the subject polynucleotides.

A subject library can comprise from about 10 individual members to about 10¹² individual members; e.g., a subject library can comprise from about 10 individual members to about 10² individual members, from about 10² individual members to about 10³ individual members, from about 10³ individual members to about 10⁵ individual members, from about 10⁵ individual members to about 10⁷ individual members, from about 10⁷ individual members to about 10⁹ individual members, or from about 10⁹ individual members to about 10¹² individual members.

In certain embodiments, two of the vectors differ in the encoded polynucleotides in their respective DNA-targeting sequences, Cas effector enzyme-binding sequences, and/or the copy number, orientation, identity (e.g., sequence, or binding specificity) of the recombinase/integrase/transposase recognition sequences.

For example, in certain embodiments, an “individual member” of a subject library differs from other members of the library in the nucleotide sequence of the DNA-targeting sequence of the subject polynucleotide. Thus, e.g., each individual member of a subject library can comprise the same or substantially the same nucleotide sequence of the Cas effector enzyme-binding sequence as all other members of the library; and can comprise the same or substantially the same nucleotide sequence of the recombinase/integrase/transposase recognition sequence as all other members of the library; but differs from other members of the library in the nucleotide sequence of the DNA-targeting sequence of the subject polynucleotide. In this way, the library can comprise members that bind to different target polynucleotide sequences that are either on the same target gene or on different target genes.

In a related embodiment, members of the library may differ such that different DNA-targeting sequences are associated with different recombinase/integrase/transposase recognition sequences, such that different target DNA can be independently modified for insertion of different donor sequences flanked by different recombinases/integrases/transposases.

### 9. Kits

The present invention also provides a kit for carrying out a subject method. The may comprise one or more (e.g., two or more) of: (1) a subject polynucleotide, or a vector encoding the same; (2) a second vector encoding the CRISPR/Cas effector enzyme, such as Cas9 or Cpf1 (e.g., wt or nickase), or fusions thereof with one or more of recombinase/integrase/reverse transcriptase; (3) optionally a third vector encoding an integrase or a recombinase, to the extent that integrase or recombinase is not fused to the Cas effector enzyme; and (4) a donor sequence flanked by integrase or recombinase recognition sequences, optionally on a fourth vector.

In certain embodiments, one or more of the first to the fourth vectors, if present, are formulated for delivery to a cell, either in vitro or in vivo.

In certain embodiments, the kit may further comprise transformation, transfection, or infection reagents to facilitate the introduction of the vectors, fusion proteins, or recombinase/integrase into the cell.

In certain embodiments, any two or more of (1)-(4) may be encoded by the same vector.

In certain embodiments, the kit may further comprise one or more buffers or reagents that facilitate the introduction of any one of (1)-(4) into a host cell, such as reagents for transformation, transfection, or infection.

For example, a subject kit can further include one or more additional reagents, where such additional reagents can be selected from: a buffer; a wash buffer; a control reagent; a control expression vector or RNA polynucleotide; a reagent for in vitro production of the wt or nickase of the Cas effector enzymes or fusions thereof from DNA; and the like.

Components of a subject kit can be in separate containers; or can be combined in a single container.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

### Example 1 Targeted Insertion of Donor DNA Payload by Combining Prime Editing and Bxb1 Integrase-mediated Recombination

This example demonstrates that a large 2.6 kb GOIs (a selection marker and a fluorescent reporter gene in this example) can be inserted site-specifically into a target genomic locus in a target cell, using the methods and reagents of the invention, specifically, through Bxb1 integrase-mediated recombination.

To target HEK3 locus for inserting a donor DNA payload containing CAGGS promoter-driven Blasticidin resistance gene-2A-mScarlet fluorescent gene-BGH polyA signal (CAGGS-Blast-2A-mScarlet-pA, 2605 bp), a donor DNA vector with the GOIs flanked by Bxb1-specific attB sites was created. The GOIs include the coding sequences for the Blasticidin resistance gene-2A (Blast-2A) and the mScarlet fluorescent gene (mScarlet). A CAGGS promoter drives the transcription of the GOIs. The GOIs were also flanked by the AttB(gt)(-) (SEQ ID NO: 1) and AttB(ga)(-) (SEQ ID NO: 1) Bxb1 integrase recognition sequences.

> AttB(gt) = AttB(Bxb1) wildtype sequence with gt
central dinucleotide:
(SEQ ID NO: 1)
TCGGCCGGCTTGTCGACGACGgcggtctcCGTCGTCAGGATCATCCGGGC
> AttB(ga) = AttB(Bxb1-ga) mutant site with ga
central dinucleotide:
(SEQ ID NO: 2)
TCGGCCGGCTTGTCGACGACGgcggactcCGTCGTCAGGATCATCCGGGC

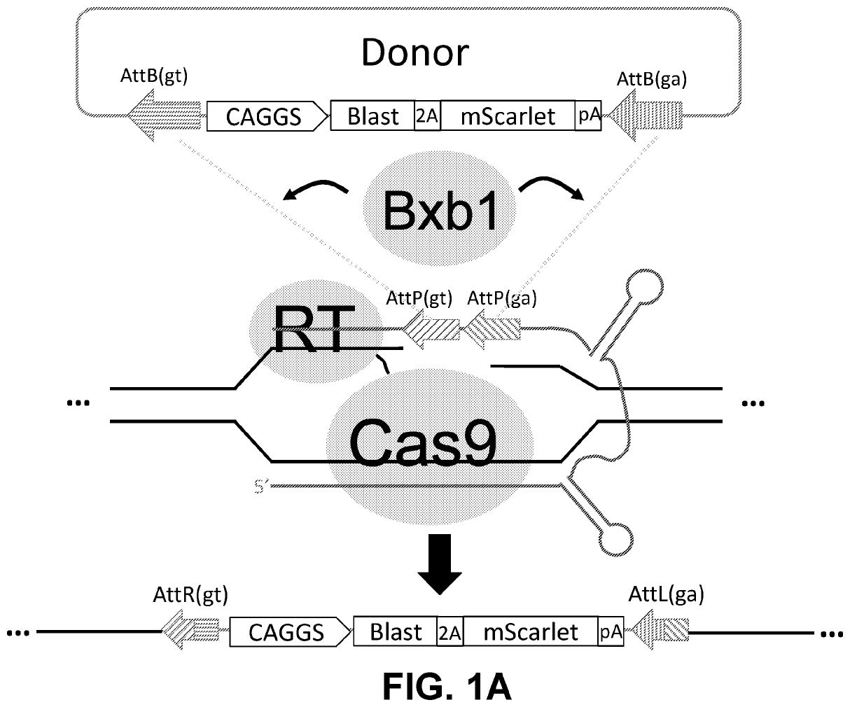

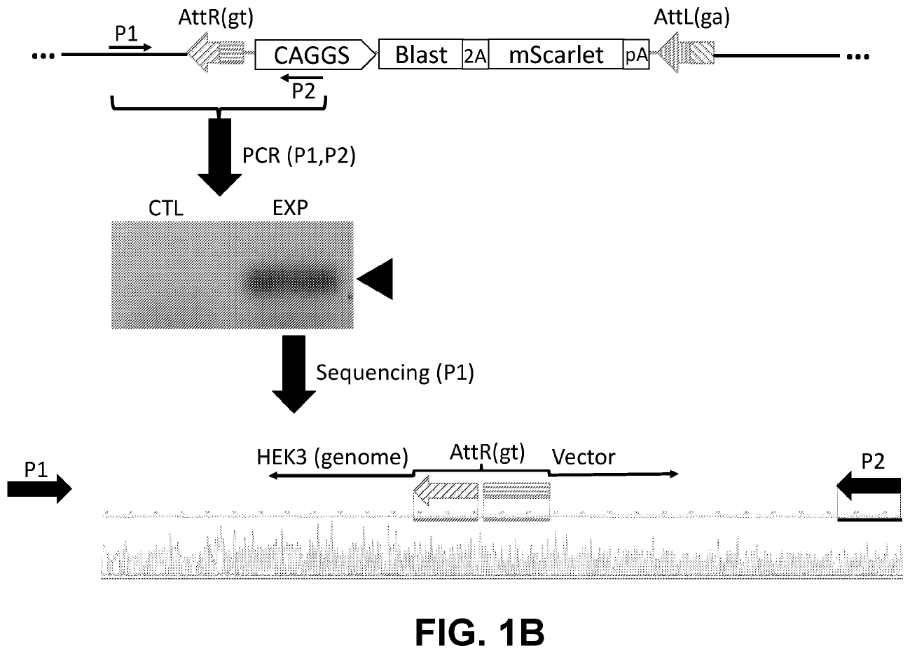

Thus the donor sequence is AttB(gt)(-)-CAGGS-Blast-2A-mScarlet-pA-AttB(ga)(-), the GOI payload was flanked by AttB(gt) and AttB(ga) sites in reverse orientation (FIG. 1A). Orthogonal Bxb1 sites were used here to ensure unidirectional insertion.

Meanwhile, a vector expressing pegRNA was constructed (SEQ ID NO: 3) with priming site for HEK3 locus, and reverse transcription (RT) template encoding cognate AttP(gt) (SEQ ID NO: 4) and AttP(ga) (SEQ ID NO: 5) sites (FIG. 1A).

> pegRNA-HEK3_Bxb1ga-Bxb1_attP (SEQ ID NO: 3)
ggcccagactgagcacgtgagtttAagagctaTGCTGGAAACAGCAtagca
agttTaaataaggctagtccgttatcaacttgaaaaagtggcaccgagtcg
gtgctctgccatcaGTCGTGGTTTGTCTGGTCAACCACCgcggactcAGTG
GTGTACGGTACAAACCCCGACgagttggtcgtcgtaccgtaTCGTGGTTTG
TCTGGTCAACCACCGCGgtCTCAGTGGTGTACGGTACAAACCCcgtgctca
gtctgttttttt
> AttP(gt) = AttP(Bxb1) wildtype sequence with gt
central dinucleotide (SEQ ID NO: 4):
TCGTGGTTTGTCTGGTCAACCACCGCGgtCTCAGTGGTGTACGGTACAAAC
CC
> AttP(ga) = AttP(Bxb1-ga) mutant site with ga
central dinucleotide (SEQ ID NO: 5):
GTCGTGGTTTGTCTGGTCAACCACCgcggactcAGTGGTGTACGGTACAAA
CCCCGAC

Bxb1-expressing plasmid (pCMV-Bx, Addgene #51552), pegRNA-expressing plasmid, and the donor DNA vector (FIG. 1A). The PE2 vector encodes a Cas9 nickase-reverse transcriptase fusion. The Cas9 nickase has the H840A mutation, thus lacking the HNH nuclease activity. The reverse transcriptase is the M-MLV RT from the Moloney murine leukemia virus with multiple mutations (D200N, T306K, W313F, T330P, L603W).

To detect the targeted insertion of the DNA payload, transfected HEK293T cells were harvested 48 hours after transfection, and genomic DNA was then extracted. Genoptying PCR using primer pairs P1, P2 was conducted to verify correct integration (FIG. 1B).

The P1 primer (SEQ ID NO: 6) and P2 primer (SEQ ID NO: 7) anneal to HEK3 genomic DNA and payload DNA, respectively. Thus PCR amplification was only possible from payload DNA integrated into the target genomic DNA.

>P1
(SEQ ID NO: 6)
ATGTGGGCTGCCTAGAAAGG
>P2
(SEQ ID NO: 7)
TTGGACATGAGCCAATATAAATG

Sample transfected with the full set of plasmids (EXP: experimental sample) showed a band indicative of the insertion with the predicted size, while control sample (CTL) transfected with a pegRNA without AttP sites (SEQ ID NO: 8) is negative for the PCR (FIG. 1B).

> pegRNA without AttP sites as negative control
(SEQ ID NO: 8)
ggcccagactgagcacgtgagtttAagagctaTGCTGGAAACAGCAtagca
agttTaaataaggctagtccgttatcaacttgaaaaagtggcaccgagtcg
gtgctctgccatcaaagcgtgctcagtctgttttttt

Sequencing of the positive band from the experimental sample confirmed the expected HEK3 genomic sequence, recombined AttR sequence, as well as vector sequence, indicative of the precise insertion of the DNA payload into the designed insertion point (FIG. 1B).

### Example 2 Targeted Insertion of Donor DNA Payload by Combining Prime Editing and Dual Integrase-Mediated Recombination

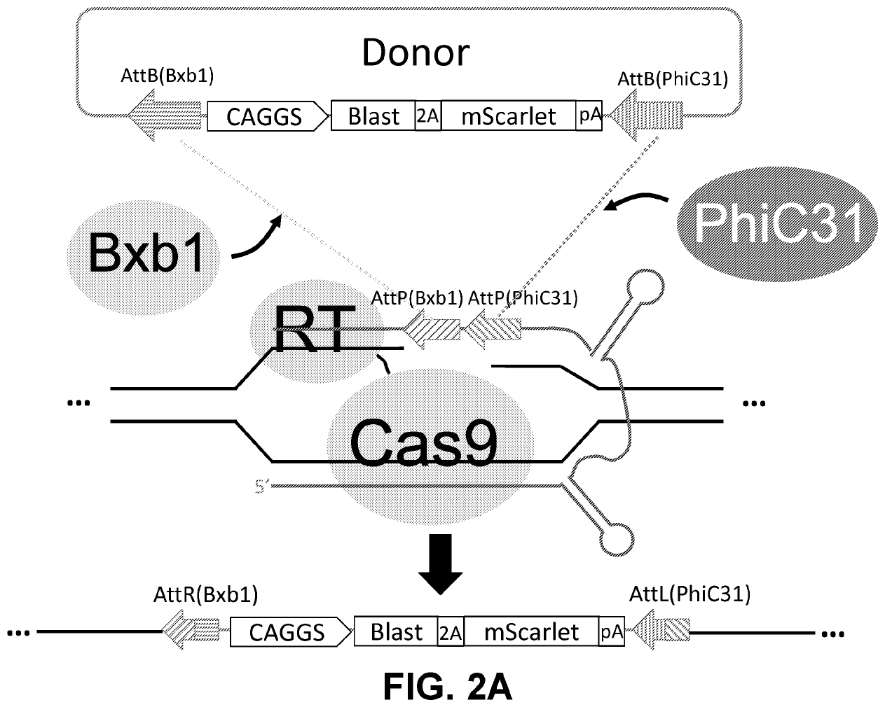

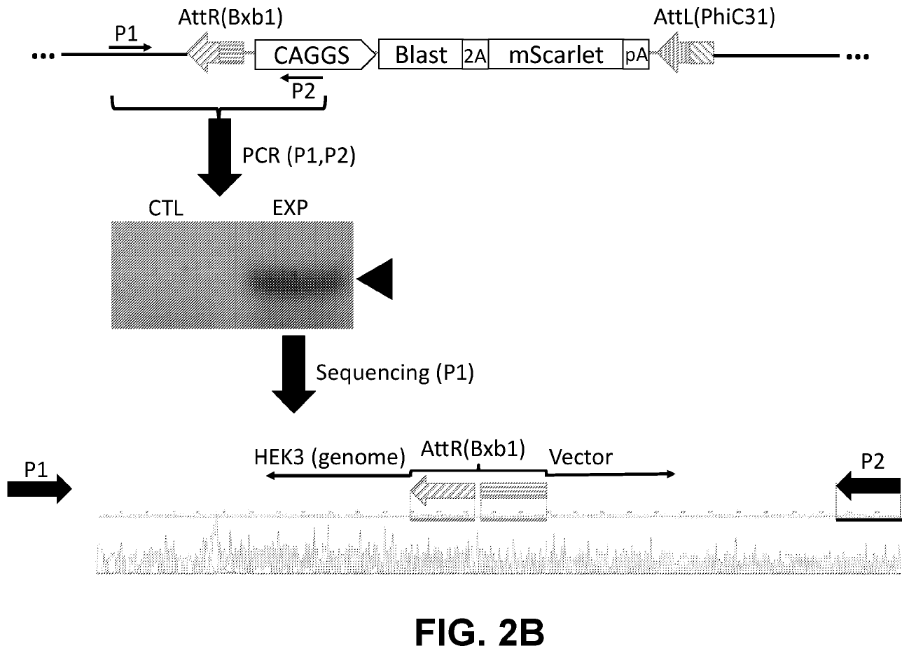

As in Example 1, the HEK3 locus was targeted for inserting a donor DNA payload comprising a CAGGS promoter-driven Blasticidin resistance gene-2A-mScarlet fluorescent gene-BGH polyA signal (CAGGS-Blast-2A-mScarlet-pA, 2605 bp). Specifically, a donor DNA was first created to contain the payload (GOI) (CAGGS-Blast-2A-mScarlet-pA) flanked by the AttB(Bxb1) (SEQ ID NO: 1) and AttB(PhiC31) (SEQ ID NO: 9) sites in reverse orientation —AttB(Bxb1)(-)-CAGGS-Blast-2A-mScarlet-pA-AttB(PhiC31)(-) (FIG. 2A).

> AttB(PhiC31)
(SEQ ID NO: 9)
GTGCGGGTGCCAGGGCGTGCCCTTGGGCTCCCCGGGCGCGTACTCC

Orthogonal integrases and their sites were used to ensure unidirectional insertion.

Meanwhile, a vector was constructed to express a pegRNA (SEQ ID NO: 10) with a priming site for the HEK3 locus, and RT template encoding the cognate AttP(Bxb1) (SEQ ID NO: 4) and AttP(PhiC31) (SEQ ID NO: 11) sites (FIG. 2A).

> pegRNA-HEK3_PhiC1-Bxb1_attP
(SEQ ID NO: 10)
ggcccagactgagcacgtgagtttAagagctaTGCTGGAAACAGCAtagca
agttTaaataaggctagtccgttatcaacttgaaaaagtggcaccgagtcg
gtgctctgccatcaCCCAGGTCAGAAGCGGTTTTCGGGAGTAGTGCCCCAA
CTGGGGTAACCTTTGAGTTCTCTCAGTTGGGGGCGTAGGGTCGCCGACATG
ACACAATACAAACCCcgtgctcagtctgttttttt
> AttP(PhiC31)
(SEQ ID NO: 11)
CCCAGGTCAGAAGCGGTTTTCGGGAGTAGTGCCCCAACTGGGGTAACCTTT
GAGTTCTCTCAGTTGGGGGCGTAGGGTCGCCGACATGACACAAGGGGTT

HEK293T cells were then transfected with Prime editor 2 (pCMV-PE2, Addgene #132775, see above), Bxb1-expressing plasmid (pCMV-Bx, Addgene #51552), PhiC31-expression plasmid (pCS-kI, Addgene #51553), pegRNA-expressing plasmid, and the donor DNA vector (FIG. 2A).

To detect the targeted insertion of the DNA payload, cells were harvested 48 hours after transfection. Genomic DNA was extracted, and genoptying PCR was conducted using primer pairs P1, P2 (FIG. 2B). P1 (SEQ ID NO: 6) and P2 (SEQ ID NO: 7) anneals to HEK3 genomic DNA and payload DNA, respectively. Thus only cells with correctly integrated payload (GOI) DNA can produce the desired PCR product.

The results show that, sample transfected with the full set of plasmids (EXP: experimental sample) produced a band indicative of the designed genomic insertion. Meanwhile, control sample (CTL) transfected with a pegRNA without the AttP site (SEQ ID NO: 8) is negative for the PCR (FIG. 2B). Sequencing of the positive band from the experimental sample confirmed the expected HEK3 genomic sequence, recombined AttR sequence, as well as the vector sequence indicative of the precise insertion of the DNA payload (FIG. 2B).

### Example 3 Targeted Insertion of Donor DNA Payload by Combining Prime Editing and FlpE Recombinase

As in Example 1, HEK3 locus was targeted for inserting a donor DNA payload comprising a CAGGS promoter-driven Blasticidin resistance gene-2A-mScarlet fluorescent gene-BGH polyA signal (CAGGS-Blast-2A-mScarlet-pA, 2605 bp). Specifically, a donor DNA was first created to contain the payload (GOI) (CAGGS-Blast-2A-mScarlet-pA) flanked by two FRT (SEQ ID NO: 12) sites in reverse orientation—FRT(-)-CAGGS-Blast-2A-mScarlet-pA-FRT(-) (FIG. 3).

> FRT
(SEQ ID NO: 12)
GAAGTTCCTATTCcGAAGTTCCTATTCtctagaaaGtATAGGAACTTC

A vector was constructed to express a pegRNA (SEQ ID NO: 13) with a priming site for the HEK3 locus, and RT template encoding one FRT (SEQ ID NO: 12) site (FIG. 3A).

> pegRNA-HEK3_FRT
(SEQ ID NO: 13)
ggcccagactgagcacgtgagtttAagagctaTGCTGGAAACAGCAtagca
agttTaaataaggctagtccgttatcaacttgaaaaagtggcaccgagtcg
gtgctctgccatcaGAAGTTCCTATTCcGAAGTTCCTATTCtctagaaaGt
ATAGGAACTTCcgtgctcagtctgttttttt

HEK293T cells were transfected with Prime editor 2 (pCMV-PE2, Addgene #132775, see above), FlpE-expressing plasmid (pCAGGS-FlpE-puro, Addgene #20733), pegRNA-expressing plasmid, and the donor DNA vector (FIG. 3A).

To detect the targeted insertion of the DNA payload, cells were harvested 48 hours after transfection. Genomic DNA was extracted, and genoptying PCR was conducted using primer pairs P1, P2 (FIG. 3B). P1 (SEQ ID NO: 6) and P2 (SEQ ID NO: 7) anneals to HEK3 genomic DNA and payload DNA, respectively. Thus only cells with correctly integrated payload (GOI) DNA can produce the desired PCR product.

Sequencing of the PCR band from the experimental sample confirmed the expected HEK3 genomic sequence, the FRT sequence, as well as the vector sequence indicative of the precise insertion of the DNA payload (FIG. 3B).

### REFERENCES

1. Khan, F. A. et al. CRISPR/Cas9 therapeutics: a cure for cancer and other genetic diseases. Oncotarget 7, 52541-52552 (2016). doi:10.18632/oncotarget.9646. PMC5239572
2. Xiong, X., Chen, M., Lim, W. A., Zhao, D. & Qi, L. S. CRISPR/Cas9 for Human Genome Engineering and Disease Research. Annual review of genomics and human genetics 17, 131-154 (2016). doi:10.1146/annurev-genom-083115-022258.
3. Wiles, M. V., Qin, W., Cheng, A. W. & Wang, H. CRISPR-Cas9-mediated genome editing and guide RNA design. Mammalian genome: official journal of the International Mammalian Genome Society 26, 501-510 (2015). doi:10.1007/s00335-015-9565-z. PMC4602062
4. Anzalone, A. V. et al. Search-and-replace genome editing without double-strand breaks or donor DNA. Nature 576, 149-157 (2019). doi:10.1038/s41586-019-1711-4. PMC6907074
5. Olorunniji, F. J. et al. Multipart DNA Assembly Using Site-Specific Recombinases from the Large Serine Integrase Family. Methods in molecular biology (Clifton, N.J.) 1642, 303-323 (2017). doi:10.1007/978-1-4939-7169-5_19.
6. Beard, C., Hochedlinger, K., Plath, K., Wutz, A. & Jaenisch, R. Efficient method to generate single-copy transgenic mice by site-specific integration in embryonic stem cells. Genesis 44, 23-28 (2006). doi:10.1002/gene.20180.
7. Zhu, F. et al. DICE, an efficient system for iterative genomic editing in human pluripotent stem cells. Nucleic Acids Res 42, e34 (2014). doi:10.1093/nar/gkt1290. PMC3950688
8. Araki, K., Araki, M. & Yamamura, K. Targeted integration of DNA using mutant lox sites in embryonic stem cells. Nucleic Acids Res 25, 868-872 (1997). doi:10.1093/nar/25.4.868. PMC146486
9. Beard, C., Hochedlinger, K., Plath, K., Wutz, A. & Jaenisch, R. Efficient method to generate single-copy transgenic mice by site-specific integration in embryonic stem cells. Genesis 44, 23-28 (2006).

All references cited herein are incorporated herein by reference.

1. A polynucleotide comprising:
(1) a DNA-targeting sequence that is complementary to the target strand (TS) of a double-stranded target DNA sequence, the target strand is complementary to the non-target strand (NTS) of the double-stranded target DNA sequence;
(2) a binding sequence for a CRISPR-Cas system effector enzyme;
(3) an integrase or recombinase recognition sequence or complement thereof; and,
(4) a primer binding sequence complementary to a primer sequence.

2. The polynucleotide of claim 1, wherein the CRISPR-Cas system effector enzyme is a Type II or Type V Class II CRISPR-Cas system effector enzyme.

3. The polynucleotide of claim 1 or 2, wherein the primer sequence comprises the 3′ end resulting from the cleavage of the non-target strand by (e.g., the RuvC or RuvC-like nuclease activity of) the effector enzyme, and/or when the polynucleotide is complexed with the effector enzyme via the binding sequence and guides the cleavage of the non-target strand by (e.g., the RuvC or RuvC-like nuclease activity of) the effector enzyme.

4. A pegRNA (primer editing guide RNA) comprising a recombinase or integrase recognition sequence (or complement thereof), wherein the recombinase or integrase recognition sequence (or complement thereof) is positioned on the pegRNA for insertion into a target DNA of the pegRNA.

5. The polynucleotide of any one of claims 1-4, wherein the effector enzyme is a Type II Class II CRISPR-Cas system effector enzyme.

6. The polynucleotide of claim 5, wherein the effector enzyme is a Cas9.

7. The polynucleotide of claim 6, wherein the Cas9 is SpCas9 from Streptococcus pyogenes, SaCas9 from Staphylococcus aureus, StCas9 from Streptococcus thermophilus, NmCas9 from Neisseria meningitidis, FnCas9 from Francisella novicida, CjCas9 from Campylobacter jejuni, ScCas9 from Streptococcus canis, or a variant thereof (such as eSpCas9, SpCas9-HF1, and xCas9).

8. The polynucleotide of any one of claims 1-7, wherein the effector enzyme lacks HNH nuclease activity.

9. The polynucleotide of any one of claims 1-4, wherein the effector enzyme is a Type V Class II CRISPR-Cas system effector enzyme that lacks HNH nuclease activity.

10. The polynucleotide of claim 9, wherein the effector enzyme is Cpf1, C2c1, or C2c3, optionally, the effector enzyme is a nickase with only RuvC nuclease activity.

11. The polynucleotide of any one of claims 1-10, wherein the recombinase is a Cre recombinase, a Hin recombinase, a Tre recombinase, or an FLP recombinase.

12. The polynucleotide of any one of claims 1-10, wherein the integrase is a phage-encoded serine integrase (such as R4, φC31, φBT1, Bxb1, SPBc, TP901-1, Wβ, FC1, φK38, RV, A118, BL3, MR11, TG1 and φ370).

13. The polynucleotide of any one of claims 1-10, wherein the integrase is a transposase (such as Tn3, Tn5, Tn7, piggyBac, SleepingBeauty, or mos1).

14. The polynucleotide of claim 12, wherein the integrase is Bxb1 or YC31.

15. The polynucleotide of any one of claims 1-14, which is an RNA.

16. The polynucleotide of any one of claims 1-15, wherein the DNA-targeting sequence is about 11-13 bases in length, about 14-20 bases in length, about 21-72 bases in length, or about 32-38 bases in length.

17. The polynucleotide of any one of claims 1-15, wherein the DNA-targeting sequence is complementary to the target strand of the double-stranded target DNA sequence over about 12-22 nucleotides (nts), about 14-20 nts, about 16-20 nts, about 18-20 nts, or about 12, 14, 16, 18, or 20 nts (preferably, the complementary region comprises a continuous stretch of 12-22 nts, preferably at the 3′ end of the DNA-binding sequence).

18. The polynucleotide of any one of claims 1-17, wherein the DNA-targeting sequence is at least about 60%, 70%, 80%, 85%, 90%, 95% or more complementary to the target strand.

19. The polynucleotide of any one of claims 1-18, wherein the DNA-targeting sequence has a 5′ end nucleotide G.

20. The polynucleotide of any one of claims 1-19, further comprising a linker sequence linking the DNA-targeting sequence to the binding sequence.

21. The polynucleotide of any one of claims 1-20, wherein the binding sequence comprises a hairpin structure.

22. The polynucleotide of any one of claims 1-21, wherein the binding sequence is about 37-47 nt, or about 42 nt.

23. The polynucleotide of any one of claims 1-22, wherein the integrase or recombinase recognition sequence comprises recognition sequence for two different integrases or recombinases.

24. The polynucleotide of any one of claims 1-23, wherein the integrase or recombinase recognition sequence comprises any one of SEQ ID NOs: 1, 2, 4, 5, 9, 11, or 12.

25. The polynucleotide of any one of claims 1-24, wherein the complementary sequence is at least about 6, 8, 10, 12, 14, 15, 16, 18, or 20 bases in length.

26. The polynucleotide of any one of claims 1-25, wherein the complementary sequence is at the 3′ end of the polynucleotide.

27. The polynucleotide of any one of claims 1-26, wherein the target DNA sequence is at, within, or adjacent to a target gene of interest (GOI).

28. The polynucleotide of claim 27, wherein the GOI is a defective gene, a disease gene, or a wild-type or mutant gene desired to be inactivated.

29. The polynucleotide of claim 27 or 28, wherein the target DNA sequence is within the first intron of the GOI.

30. The polynucleotide of any one of claims 1-26, wherein the target DNA sequence comprises or is adjacent to a transcription regulatory element.

31. The polynucleotide of claim 30, wherein the transcription regulatory element comprises one or more of: core promoter, proximal promoter element, enhancer, silencer, insulator, and locus control region.

32. The polynucleotide of any one of claims 1-26, wherein the target DNA sequence comprises or is adjacent to a telomere sequence, a centromere, or a repetitive genomic sequence.

33. The polynucleotide of any one of claims 1-26, wherein the target DNA sequence comprises or is adjacent to a genomic marker sequence (or a genomic locus of interest).

34. A vector encoding the polynucleotide of any one of claims 1-33.

35. The vector of claim 34, wherein transcription of the polynucleotide is under the control of a constitutive promoter, or an inducible promoter.

36. The vector of claim 34 or 35, wherein the vector is active in a cell from a mammal (a human; a non-human primate; a non-human mammal; a rodent such as a mouse, a rat, a hamster, a Guinea pig; a livestock mammal such as a pig, a sheep, a goat, a horse, a camel, cattle; or a pet mammal such as a cat or a dog); a bird, a fish, an insect, a worm, a yeast, or a bacterium.

37. A plurality of vectors of any one of claims 34-36, wherein two of the vectors differ in the encoded polynucleotides in their respective DNA-targeting sequences, binding sequences, integrase or recombinase recognition sequences, and/or complementary sequences.

38. A complex comprising:
(a) the polynucleotide of any one of claims 1-33, and,
(b) a fusion protein comprising:
(i) a Type II or Type V Class II CRISPR-Cas system effector enzyme lacking HNH nuclease activity; and,
(ii) a reverse transcriptase;
wherein the polynucleotide is complexed with the effector enzyme lacking HNH nuclease activity through the binding sequence.

39. The complex of claim 38, further comprising:
(c) a double-stranded target DNA sequence comprising a target strand and a complementary non-target strand;
wherein the DNA-targeting sequence of the polynucleotide binds to the target strand;
and,
wherein the effector enzyme is capable of cleaving the non-target strand through the RuvC nuclease activity to release the 3′-end of the primer sequence, for priming reverse transcription by the reverse transcriptase, upon binding of the primer sequence to the complementary sequence to integrate the integrase or recombinase recognition sequence into the reverse transcription transcript.

40. The complex of claim 38 or 39, wherein the fusion protein further comprises a nuclear localization sequence (NLS).

41. The complex of any one of claims 38-40, wherein the fusion protein further comprises a recombinase or an integrase.

42. The complex of any one of claims 38-41, wherein the effector enzyme is a Cas9 nickase that lacks HNH endonuclease activity due to a point mutation at the endonuclease catalytic site of the HNH endonuclease.

43. The complex of claim 42, wherein the Cas9 nickase point mutation is H840A or equivalent thereof.

44. A host cell comprising the vector of any one of claims 34-36, or the plurality of vectors of claim 37.

45. The host cell of claim 44, wherein the vector further encodes the fusion protein of any one of claims 38-43.

46. The host cell of claim 44, further comprising a second vector encoding the fusion protein of any one of claims 38-43.

47. The host cell of claim 45 or 46, wherein expression of the fusion protein is under the control of a constitutive promoter or an inducible promoter.

48. The host cell of any one of claims 44-47, further comprising a donor sequence flanked by compatible integrase or recombinase recognition sequences that can direct the integration of the donor sequence into target DNA sequence comprising the integrase or recombinase recognition sequences, wherein the donor sequence is at least about 100 bp, 150 bp, 200 bp, 500 bp, 1 kb, 1.5 kb, 2, kb, 3 kb, 4 kb, 5 kb, 6 kb, 8 kb, or 10 kb.

49. The host cell of any one of claims 44-48, which is in a live animal.

50. The host cell of any one of claims 44-48, which is a cultured cell.

51. A method of integrating a donor sequence into a target DNA sequence, the method comprising:
(1) providing a complex of any one of claims 38-43 (and the target DNA sequence if necessary), the integrase or recombinase (if necessary), and the donor sequence flanked by compatible integrase or recombinase recognition sequences;
(2) allowing editing of the target DNA sequence to generate an edited target DNA sequence, by primer extension from the primer sequence, in order to insert integrase or recombinase recognition sequence according to the integrase or recombinase recognition sequence on the polynucleotide of any one of claims 1-33; and,
(3) allowing insertion of the donor sequence into the edited target DNA sequence via site-specific recombination.

52. The method of claim 51, wherein the complex is assembled inside a cell; wherein the target DNA sequence is a part of the genomic DNA of the cell; and wherein the polynucleotide of any one of claims 1-33, the vector of claims 34-37, the fusion protein of any one of claims 38-43 or a polynucleotide encoding the fusion protein, and the donor sequence flanked by compatible integrase or recombinase recognition sequences are introduced into the cell.

53. The method of claim 51 or 52, wherein the target DNA sequence is at, within, or adjacent to a target gene of interest (GOI).

54. The method of claim 53, wherein the GOI is a defective gene, a disease gene, or a wild-type or mutant gene desired to be inactivated.

55. The method of claim 53 or 54, wherein the target DNA sequence is within the first intron of the GOI.

56. A kit comprising one or more of:
(1) a polynucleotide of any one of claims 1-33, or a vector of any one of claims 34-37;
(2) a second vector encoding a fusion protein of any one of claims 38-43, or the fusion protein of any one of claims 38-43 formulated for delivery to a cell; optionally comprising in the same or different packaging a third vector encoding an integrase or a recombinase, or the integrase or recombinase formulated for delivery to the cell, and
(3) a donor sequence flanked by integrase or recombinase recognition sequences.

57. The kit of claim 56, further comprising transformation, transfection, or infection reagents to facilitate the introduction of the vectors, fusion protein, or recombinase/integrase into the cell.
